(19) United States
(12) Patent Application Publication
(10) Pub. No.: US 2026/0041913 A1
(43) Pub. Date: Feb. 12, 2026

(54) DETERMINING LOCATION OF MEDICAL LEAD DURING IMPLANT

(71) Applicant: Medtronic, Inc., Minneapolis, MN (US)

(72) Inventors: Todd D. Zenisek, Georgetown, TX (US); Juan G. Hincapie, Maple Grove, MN (US); Andrew J. Cleland, St. Paul, MN (US); Leonid M. Litvak, Los Angeles, CA (US); Andrew L. Schmeling, Holmen, WI (US)

(21) Appl. No.: 18/998,860

(22) PCT Filed: Jul. 24, 2023
(86) PCT No.: PCT/US2023/028433

§ 371 Date: Jan. 27, 2025

Related U.S. Application Data
(60) Provisional application No. 63/369,725, filed on Jul. 28, 2022.

(51) Int. Cl. A61N 1/36; A61B 5/00; A61B 5/06; A61N 1/05

(52) U.S. Cl. CPC A61N 1/36071; A61B 5/065; A61B 5/4836; A61N 1/0551; A61N 1/36135

(57) ABSTRACT

Devices, systems, and techniques for determining medical lead placement are described. In one example, a system includes sensing circuitry configured to detect, via a lead, a bioelectric signal of the patient, wherein the lead comprises one or more electrodes, and wherein the lead is configured to be located in an epidural space of a patient and displaced laterally from a dorsal horn of the patient. Additionally, the system includes processing circuitry configured to determine, based on the bioelectric signal, a proximity of each electrode of the one or more electrodes to a dorsal root of the patient; and output information indicative of the proximity of each electrode of the one or more electrodes to the dorsal root.

This application claims the benefit of U.S. Provisional Ser. No. 63/369,725, filed on Jul. 28, 2022, the entire content of which is incorporated herein by reference.

### TECHNICAL FIELD

This disclosure generally relates to electrical stimulation therapy.

### BACKGROUND

Medical devices may be external or implanted and may be used to deliver electrical stimulation therapy to patients via various tissue sites to treat a variety of symptoms or conditions such as chronic pain, tremor, Parkinson's disease, epilepsy, urinary or fecal incontinence, sexual dysfunction, obesity, or gastroparesis. A medical device may deliver electrical stimulation therapy via one or more leads that include electrodes located proximate to target locations associated with the brain, the spinal cord, pelvic nerves, peripheral nerves, or the gastrointestinal tract of a patient. Stimulation proximate the spinal cord, proximate the sacral nerve, within the brain, and proximate peripheral nerves are often referred to as spinal cord stimulation (SCS), sacral neuromodulation (SNM), deep brain stimulation (DBS), and peripheral nerve stimulation (PNS), respectively.

### SUMMARY

In general, systems, devices, and techniques are described for sensing and analyzing bioelectric signals to determine a location for delivering electrical stimulation, such as a location for implanting a medical lead and/or selecting an electrode combination for electrical stimulation. SCS systems may stimulate a spinal cord of a patient to mitigate or completely eliminate pain experienced by the patient. Dorsal roots may enter the spinal cord of the patient at one or more locations along a length of the spinal cord. Pain experienced by the patient may be caused by pain signals entering the spinal cord via one or more dorsal roots. It may be beneficial to identify the one or more dorsal roots carrying pain signals, and place one or more medical leads proximate to the dorsal roots carrying pain signals so that the one or more medical leads can deliver electrical stimulation to mitigate or completely eliminate the pain experienced by the patient. In some examples, a system may identify the one or more dorsal roots carrying pain signals, or other non-pain signals, by detecting bioelectric signals of the patient. Bioelectric signals may, in some cases, include local field potentials (LFPs) and evoked compound action potentials (ECAPs). The system may output information indicative of a proximity of each electrode relative to a dorsal root carrying pain signals or non-pain signals that may be associated with patient pain or discomfort. This information may enable a clinician to implant the medical lead and/or choose electrode combinations more effectively as compared with systems that do not output information indicating the proximity of electrodes to nerve tissue.

An example system may include a medical lead located in a lateral region of the epidural space. In some cases, the lateral region of the epidural space may be proximate to one or more locations at which a dorsal root connects with the spinal cord. In some examples, a system including a medical lead placed in the lateral region of the epidural space may consume a smaller amount of energy (e.g., lower amplitudes and/or shorter pulse widths) as compared with systems that include a medical lead placed in a central region of the epidural space. When a lead is located in the lateral region of the epidural space, the electrodes of the lead may be closer to nerve tissue as compared with medical leads placed in a central region of the epidural space. Since the lead in the lateral region is closer to nerve tissue, the lead in the lateral region may require less energy to stimulate the nerve tissue as compared with medical leads placed in the central region of the epidural space which may be further from the target nerves.

In some examples, a system includes sensing circuitry configured to detect, via a lead, a bioelectric signal of the patient, wherein the lead comprises one or more electrodes, and wherein the lead is configured to be located in an epidural space of a patient and displaced laterally from a dorsal horn of the patient. Additionally, the system includes processing circuitry configured to determine, based on the bioelectric signal, a proximity of each electrode of the one or more electrodes to a dorsal root of the patient; and output information indicative of the proximity of each electrode of the one or more electrodes to the dorsal root.

In some examples, a method includes detecting, by sensing circuitry via a lead, a bioelectric signal of the patient, wherein the lead comprises one or more electrodes, and wherein the lead is configured to be located in an epidural space of a patient and displaced laterally from a dorsal horn of the patient; determining, by processing circuitry based on the bioelectric signal, a proximity of each electrode of the one or more electrodes to a dorsal root of the patient; and outputting, by the processing circuitry, information indicative of the proximity of each electrode of the one or more electrodes to the dorsal root.

In some examples, a computer-readable storage medium includes instructions that, when executed, cause one or more processors to: detect, via a lead, a bioelectric signal of the patient, wherein the lead comprises one or more electrodes, and wherein the lead is configured to be located in an epidural space of a patient and displaced laterally from a dorsal horn of the patient; determine, based on the bioelectric signal, a proximity of each electrode of the one or more electrodes to a dorsal root of the patient; and output information indicative of the proximity of each electrode of the one or more electrodes to the dorsal root.

The details of one or more examples of the techniques of this disclosure are set forth in the accompanying drawings and the description below. Other features, objects, and advantages of the techniques will be apparent from the description and drawings, and from the claims.

### BRIEF DESCRIPTION OF DRAWINGS

FIG. 1 is a conceptual diagram illustrating an example system that includes an implantable medical device (IMD) configured to deliver spinal cord stimulation (SCS) therapy and an external programmer, in accordance with one or more techniques of this disclosure.

FIG. 2 is a block diagram of the IMD of FIG. 1, in accordance with one or more techniques of this disclosure.

FIG. 3 is a block diagram of the example external programmer of FIG. 1, in accordance with one or more techniques of this disclosure.

FIG. 4 is a conceptual diagram illustrating a cross section of a spinal column, in accordance with one or more techniques of this disclosure.

FIG. 5 is a conceptual diagram illustrating a perspective view of an example spinal column, in accordance with one or more techniques of this disclosure.

FIG. 6 is a flow diagram illustrating an example operation for determining a position of one or more electrodes based on sensed bioelectric signals, in accordance with one or more techniques of this disclosure.

### DETAILED DESCRIPTION

The disclosure describes examples of medical devices, systems, and techniques for sensing and analyzing bioelectric signals. SCS is a type of electrical stimulation therapy. SCS may include the precise delivery of electrical energy to the spinal cord, and is part of a larger class of neuromodulation. For example, system configured to perform SCS may include one or more leads implanted in the epidural space of the patient's spinal column. A clinician may position one or more medical leads in the epidural space, or select electrodes for an electrode combination, so that the system is configured to deliver electrical stimulation for mitigating or completely eliminating pain experienced by the patient via the one or more medical leads. The location of the one or more medical leads within the epidural space may determine an amount of energy required to provide effective pain relief to the patient. Consequently, it may be beneficial for the system to generate information indicative of a location of the one or more leads while the clinician is implanting the leads. This information may assist the clinician in placing the one or more leads so that the system can deliver effective pain relief while consuming a smaller amount of energy as compared with systems that do not output information indicative of the location of leads during implant. In some examples, these techniques may be utilized after lead implantation to determine which electrodes of the implanted one or more leads should be included in an electrode configuration that delivers electrical stimulation.

In some examples, a patient's spinal column includes a collection of neurons referred to herein as the “dorsal horn.” Dorsal horn neurons may carry signals along the spinal column to and from the patient's brain. Dorsal roots enter the spinal column of the patient at one or more locations along a length of the spinal column. Dorsal roots may serve as a bridge between the dorsal horn and one or more peripheral nerves of the patient. A patient may experience pain when the dorsal horn carries pain signals to the patient's brain. In some cases, these pain signals may enter the spinal cord via one or more dorsal roots. Consequently, it may be beneficial for the system to identify, during the implant of a medical lead, one or more dorsal roots carrying pain signals during the implant of a medical lead so that the clinician can position the medical lead proximate to the one or more dorsal roots carrying pain signals. For example, the system may determine a proximity of each electrode of the one or more electrodes to a dorsal root carrying pain signals. The system may output information indicative of the proximity of each electrode of the one or more electrodes to the dorsal root carrying pain signals so that the clinician can position the medical lead. The system may determine the proximity of each electrode of the one or more electrodes based on detecting one or more bioelectric signals of the patient. In some examples, the one or more bioelectric signals may correspond to the pain signals travelling through the dorsal root to the dorsal horn. In some examples, the system may determine the proximity of electrodes to the dorsal root carrying non-pain signals. These non-pain signals may not directly carry pain information, but may be associated with patient perceived pain and thus be used by the system to mitigate perceived pain. The system may sense these non-pain signals in addition to, or instead of, the pain signals.

In some examples, a medical device may identify one or more dorsal roots carrying pain signals by sensing and analyzing local field potentials (LFPs). For example, a system may be configured to sense LFPs via electrodes placed in an epidural space of a patient. In some examples, the sensing electrodes may be placed near one or more dorsal roots. The system may be configured to analyze the sensed LFPs and determine whether the one or more dorsal roots near the sensing electrodes are carrying pain signals that cause the patient to experience a physiological state or a pain state. The system may use such analysis to determine a proximity of one or more electrodes of the medical lead to the one or more dorsal roots carrying the pain signals. The system may output information indicative of the proximity of the one or more electrodes to the one or more dorsal roots carrying pain signals. Such information may, in some cases, be used by a physician to place the medical lead so that the medical lead can deliver electrical stimulation therapy.

In some examples, a medical device may identify one or more dorsal roots carrying pain signals by sensing and Evoked Compound Action Potentials (ECAPs). ECAPs are bioelectric signals evoked by stimulation pulses. For example, a system may deliver electrical simulation comprising a sequence of pulses. One or more pulses of the sequence of pulses may elicit an ECAP detectable by the system. In some examples, ECAPs may indicate one or more characteristics of pain signals travelling through a dorsal root to the spinal cord.

The system is not limited to identifying a proximity of each electrode of one or more electrodes to a dorsal root carrying pain signals. Dorsal roots may carry signals other than pain signals (e.g., non-pain signals). The system may be configured to identify a proximity of each electrode of one or more electrodes to a dorsal root carrying a biometric signal other than a pain signal. Biometric signals carried by the dorsal root that are not pain signals may include signals that cause the patient to experience a feeling other than pain and may be associated with (e.g., occur coincidentally with otherwise linked to) pain. In other examples, the system may use the non-pain signals to identify non-pain conditions to be treated by the system.

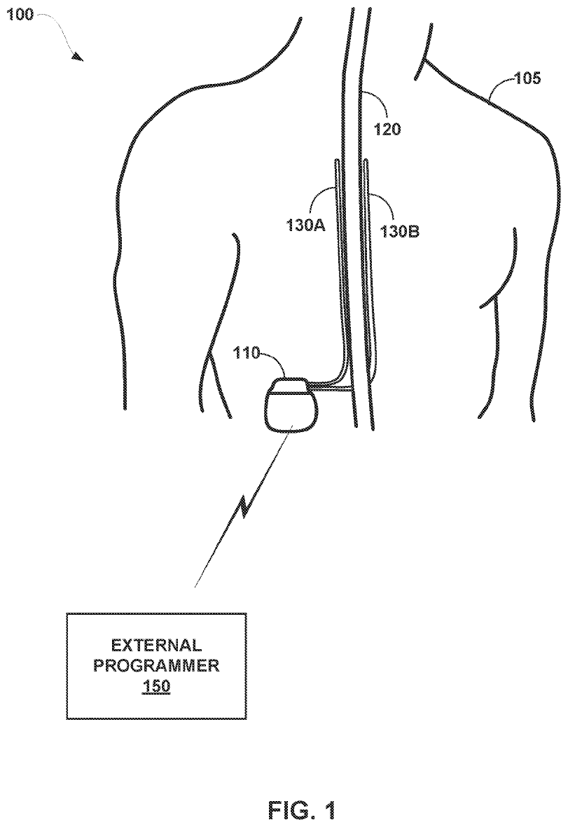

FIG. 1 is a conceptual diagram illustrating an example system 100 that includes an implantable medical device (IMD) 110 configured to deliver spinal cord stimulation (SCS) therapy and an external programmer 150, in accordance with one or more techniques of this disclosure. Although the techniques described in this disclosure are generally applicable to a variety of medical devices including external devices and IMDs, application of such techniques to IMDs and, more particularly, implantable electrical stimulators (e.g., neurostimulators) will be described for purposes of illustration. More particularly, the disclosure will refer to an implantable SCS system for purposes of illustration, but without limitation as to other types of medical devices or other therapeutic applications of medical devices.

As shown in the example of FIG. 1, system 100 includes an IMD 110, leads 130A and 130B, and external programmer 150 shown in conjunction with a patient 105, who is ordinarily a human patient. In the example of FIG. 1, IMD 110 is an implantable electrical stimulator that is configured to generate and deliver electrical stimulation therapy to patient 105 via one or more electrodes of leads 130A and/or 130B (collectively, “leads 130”), e.g., for relief of chronic pain or other symptoms. In other examples, IMD 110 may be coupled to a single lead carrying multiple electrodes or more than two leads each carrying multiple electrodes. IMD 110 may be a chronic electrical stimulator that remains implanted within patient 105 for weeks, months, or even years. In other examples, IMD 110 may be a temporary, or trial, stimulator used to screen or evaluate the efficacy of electrical stimulation for chronic therapy. In one example, IMD 110 is implanted within patient 105, while in another example, IMD 110 is an external device coupled to percutaneously implanted leads. In some examples, IMD 110 uses one or more leads, while in other examples, IMD 110 is leadless.

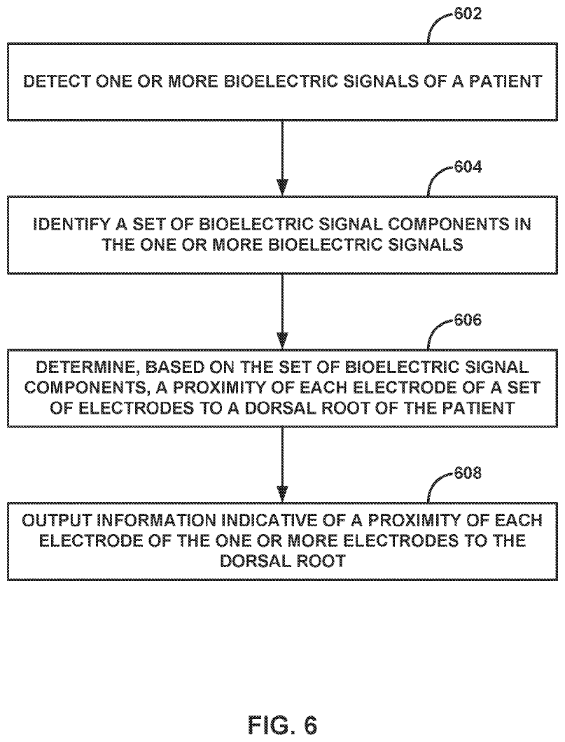

IMD 110 may be constructed of any polymer, metal, or composite material sufficient to house the components of IMD 110 (e.g., components illustrated in FIG. 6) within patient 105. In this example, IMD 110 may be constructed with a biocompatible housing, such as titanium or stainless steel, or a polymeric material such as silicone, polyurethane, or a liquid crystal polymer, and surgically implanted at a site in patient 105 near the pelvis, abdomen, or buttocks. In other examples, IMD 110 may be implanted within other suitable sites within patient 105, which may depend, for example, on the target site within patient 105 for the delivery of electrical stimulation therapy. The outer housing of IMD 110 may be configured to provide a hermetic seal for components, such as circuitry and a rechargeable or non-rechargeable power source. In addition, in some examples, the outer housing of IMD 110 is selected from a material that facilitates receiving energy to charge the rechargeable power source.

Electrical stimulation energy, which may be constant current or constant voltage-based pulses, for example, is delivered from IMD 110 to one or more target tissue sites of patient 105 via one or more electrodes (not shown) of implantable leads 130. In the example of FIG. 1, leads 130 carry electrodes that are placed adjacent to the target tissue of spinal cord 120. In examples of this disclosure, leads 130 may be placed in the epidural space of the spine of patient 105, such as near one or more of a dorsal root ganglion (DRG), dorsal roots/rootlets, dorsal root entry zone, and/or ventral roots/rootlets in order to sense LFPs and/or ECAPs. In general, leads 130 may be positioned in a lateral placement from the midline. One or more of the electrodes may be disposed at a distal tip of a lead 130 and/or at other positions at intermediate points along the lead. Leads 130 may be implanted and coupled to IMD 110. The electrodes may transfer electrical stimulation generated by an electrical stimulation generator in IMD 110 to tissue of patient 105. Although leads 130 may each be a single lead, lead 130 may include a lead extension or other segments that may aid in implantation or positioning of lead 130. In some other examples, IMD 110 may be a leadless stimulator with one or more arrays of electrodes arranged on a housing of the stimulator rather than leads that extend from the housing. In addition, in some other examples, system 100 may include one lead or more than two leads, each coupled to IMD 110 and directed to similar or different target tissue sites.

The epidural space of patient 105 may represent a space between a posterior portion of a vertebra of the patient 105 and the spinal cord 120 of the patient. The epidural space may include fat. The epidural space may extend along at least a portion of the spinal cord 120 of the patient 105 such that leads 130 may be placed in the epidural space along the spinal cord 120. Since the epidural space is proximate to the spinal cord 120, leads placed within the epidural space may stimulate the spinal cord 120 and/or other nerve tissue proximate to the spinal cord 120. The epidural space of the patient may include a central region and one or more lateral regions. The central region of the epidural space may include a center point of a cross-section of the epidural space. The lateral regions of the epidural space may be laterally displaced from the center point of the cross-section of the epidural space.

The spinal cord 120 may include a collection of neurons referred to herein as the “dorsal horn.” Dorsal horn neurons may carry signals along the spinal cord 120 to and from a brain of patient 105. Dorsal roots may enter the spinal cord 120 of the patient 105 at one or more locations along a length of the spinal cord 120. Dorsal roots may serve as a bridge between the dorsal horn and one or more peripheral nerves of the patient. In some examples, patient 105 may experience pain when the dorsal horn carries pain signals to the patient's brain. In some cases, these pain signals may enter the dorsal horn via one or dorsal roots. In some examples, a lateral region of the epidural space may be proximate to a point at which a dorsal root enters the spinal cord 120 and connects with the dorsal horn. In some examples, one or more of leads 130 may be placed in a lateral region of the epidural space of the patient 105 so that the one or more of leads 130 is proximate to one or more locations in which dorsal roots enter the spinal cord 120.

In some examples, spinal cord 120 may include one or more interneurons that relay signals between sensory neurons and motor neurons. A wind-up of one or more interneurons in the spinal cord 120 may cause patient 105 to experience pain. Wind-up may represent a phenomenon that represents a frequency-dependent response of a neuron evoked by electrical stimulation. Wind-up in one or more interneurons within spinal cord 120 may cause patient 105 to experience pain in addition to or alternatively to pain signals that enter the dorsal horn via the one or more dorsal roots. During acute pain, there may be C- and A-delta fibers transmitting pain signals from a periphery and through the lateral epidural space.

In some examples, one or more of leads 130 may be located in a central region of the epidural space (e.g., proximate to the longitudinal axis of the epidural space). The central region of the epidural space is proximate to the spinal cord 120, but a lateral region of the epidural space may be closer to a location in which a dorsal root connects with the spinal cord 120 than the central region of the epidural space.

The electrodes of leads 130 may be electrode pads on a paddle lead, circular (e.g., ring) electrodes surrounding the body of the lead, conformable electrodes, cuff electrodes, segmented electrodes (e.g., electrodes disposed at different circumferential positions around the lead instead of a continuous ring electrode), any combination thereof (e.g., ring electrodes and segmented electrodes) or any other type of electrodes capable of forming unipolar, bipolar or multipolar electrode combinations for therapy. Ring electrodes arranged at different axial positions at the distal ends of lead 130 will be described for purposes of illustration.

The deployment of electrodes via leads 130 is described for purposes of illustration, but arrays of electrodes may be deployed in different ways. For example, a housing associated with a leadless stimulator may carry arrays of electrodes, e.g., rows and/or columns (or other patterns), to which shifting operations may be applied. Such electrodes may be arranged as surface electrodes, ring electrodes, or protrusions. As a further alternative, electrode arrays may be formed by rows and/or columns of electrodes on one or more paddle leads. In some examples, electrode arrays include electrode segments, which are arranged at respective positions around a periphery of a lead, e.g., arranged in the form of one or more segmented rings around a circumference of a cylindrical lead. In other examples, one or more of leads 130 are linear leads having 8 ring electrodes along the axial length of the lead. In another example, the electrodes are segmented rings arranged in a linear fashion along the axial length of the lead and at the periphery of the lead.

The stimulation parameter set of a therapy stimulation program that defines the stimulation pulses of electrical stimulation therapy delivered by IMD 110 through the electrodes of leads 130 may include information identifying which electrodes have been selected for delivery of stimulation according to a stimulation program, the polarities of the selected electrodes, i.e., the electrode combination for the program, voltage or current amplitude, pulse frequency, pulse width, pulse shape of stimulation delivered by the electrodes. These stimulation parameters values that make up the stimulation parameter set that defines pulses may be predetermined parameter values defined by a user and/or automatically determined by system 100 based on one or more factors or user input.

In some examples, IMD 110 may deliver stimulation pulses that contribute to therapy perceived by patient 105. IMD 110 may detect ECAP signals elicited by these stimulation pulses. In other examples, stimulation pulses configured to provide therapy may prevent IMD 110 from detecting ECAP signals (e.g., because the pulse width of the stimulation pulses are long enough to interfere with propagating ECAP signals). Therefore, if control pulses (e.g., pulses that may or may not contribute to therapy) separate from informed pulses configured to provide therapy are needed to elicit a detectable ECAP signal, system 100 may employ an ECAP test stimulation program that defines stimulation parameter values that define control pulses delivered by IMD 110 through at least some of the electrodes of leads 130.

These stimulation parameter values may include information identifying which electrodes have been selected for delivery of control pulses, the polarities of the selected electrodes, i.e., the electrode combination for the program, and voltage or current amplitude, pulse frequency, pulse width, and pulse shape of stimulation delivered by the electrodes. The stimulation signals (e.g., one or more stimulation pulses or a continuous stimulation waveform) defined by the parameters of each ECAP test stimulation program are configured to evoke a compound action potential from nerves. In some examples, the ECAP test stimulation program defines when the control pulses are to be delivered to the patient based on the frequency and/or pulse width of the informed pulses. The stimulation defined by each ECAP test stimulation program may not be intended to provide or contribute to therapy for the patient, but the patient may perceive the control pulses in some examples. In addition, the ECAP test stimulation program may define the control pulses used for each sweep of pulses that are used to detect a change in an ECAP signal that is indicative of the associated lead having migrated from its original position.

Although FIG. 1 is directed to SCS therapy, e.g., therapy to treat pain, in other examples system 100 may be configured to treat any other condition that may benefit from electrical stimulation therapy. For example, system 100 may be used to treat tremor, Parkinson's disease, epilepsy, a pelvic floor disorder (e.g., urinary incontinence or other bladder dysfunction, fecal incontinence, pelvic pain, bowel dysfunction, or sexual dysfunction), obesity, gastroparesis, or psychiatric disorders (e.g., depression, mania, obsessive compulsive disorder, anxiety disorders, and the like). In this manner, system 100 may be configured to provide therapy taking the form of deep brain stimulation (DBS), peripheral nerve stimulation (PNS), peripheral nerve field stimulation (PNFS), cortical stimulation (CS), pelvic floor stimulation, gastrointestinal stimulation, or any other stimulation therapy capable of treating a condition of patient 105.

In some examples, lead 130 includes one or more sensors configured to allow IMD 110 to monitor one or more parameters of patient 105, such as patient activity, pressure, temperature, or other characteristics. The one or more sensors may be provided in addition to, or in place of, therapy delivery by lead 130.

IMD 110 is configured to deliver electrical stimulation therapy to patient 105 via selected combinations of electrodes carried by one or both of leads 130, alone or in combination with an electrode carried by or defined by an outer housing of IMD 110. The target tissue for the electrical stimulation therapy may be any tissue affected by electrical stimulation, which may be in the form of electrical stimulation pulses or continuous waveforms. In some examples, the target tissue includes nerves, smooth muscle, or skeletal muscle. In some examples, the target tissue is peripheral tissue. In the example illustrated by FIG. 1, the target tissue is tissue proximate spinal cord 120, such as within an intrathecal space or epidural space of spinal cord 120, or, in some examples, adjacent nerves that branch off spinal cord 120. Leads 130 may be introduced adjacent to spinal cord 120 in via any suitable region, such as the thoracic, cervical, or lumbar regions. Stimulation of spinal cord 120 may, for example, prevent pain signals from traveling through spinal cord 120 and to the brain of patient 105. Patient 105 may perceive the interruption of pain signals as a reduction in pain and, therefore, efficacious therapy results. In other examples, stimulation of spinal cord 120 may produce paresthesia which causes a tingling sensation that may reduce the perception of pain by patient 105, and thus, provide efficacious therapy results. In some examples, stimulation of spinal cord 120 may be configured to reduce or eliminate pain without paresthesia. IMD 110 may, in some examples, stimulate target tissue in order to decrease acute pain.

IMD 110 is configured to generate and deliver electrical stimulation therapy to a target stimulation site within patient 105 via the electrodes of leads 130 to patient 105 according to one or more therapy stimulation programs. A therapy stimulation program defines values for one or more parameters (e.g., a parameter set) that define an aspect of the therapy delivered by IMD 110 according to that program. For example, a therapy stimulation program that controls delivery of stimulation by IMD 110 in the form of pulses may define values for voltage or current pulse amplitude, pulse width, pulse rate (e.g., pulse frequency), electrode combination, pulse shape, etc. for stimulation pulses delivered by IMD 110 according to that program.

Furthermore, IMD 110 may be configured to deliver control stimulation to patient 105 via a combination of electrodes of leads 130, alone or in combination with an electrode carried by or defined by an outer housing of IMD 110 in order to detect ECAP signals (e.g., control pulses and/or informed pulses). The tissue targeted by the stimulation may be the same or similar tissue targeted by the electrical stimulation therapy, but IMD 110 may deliver stimulation pulses for ECAP signal detection via the same, at least some of the same, or different electrodes. Since control stimulation pulses can be delivered in an interleaved manner with informed pulses (e.g., when the pulses configured to contribute to therapy interfere with the detection of ECAP signals or pulse sweeps intended to detect migration of leads 130 via ECAP signals do not correspond to pulses intended for therapy purposes), a clinician and/or user may select any desired electrode combination for informed pulses. Like the electrical stimulation therapy, the control stimulation may be in the form of electrical stimulation pulses or continuous waveforms. In one example, each control stimulation pulse may include a balanced, bi-phasic square pulse that employs an active recharge phase. However, in other examples, the control stimulation pulses may include a monophasic pulse followed by a passive recharge phase. In other examples, a control pulse may include an imbalanced bi-phasic portion and a passive recharge portion. Although not necessary, a bi-phasic control pulse may include an interphase interval between the positive and negative phase to promote propagation of the nerve impulse in response to the first phase of the bi-phasic pulse. The control stimulation may be delivered without interrupting the delivery of the electrical stimulation informed pulses, such as during the window between consecutive informed pulses. The control pulses may elicit an ECAP signal from the tissue, and IMD 110 may sense the ECAP signal via two or more electrodes on leads 130. In cases where the control stimulation pulses are applied to spinal cord 120, the signal may be sensed by IMD 110 from spinal cord 120.

IMD 110 can deliver control stimulation to a target stimulation site within patient 105 via the electrodes of leads 130 according to one or more ECAP test stimulation programs. The one or more ECAP test stimulation programs may be stored in a storage device of IMD 110. Each ECAP test program of the one or more ECAP test stimulation programs includes values for one or more parameters that define an aspect of the control stimulation delivered by IMD 110 according to that program, such as current or voltage amplitude, pulse width, pulse frequency, electrode combination, and, in some examples timing based on informed pulses to be delivered to patient 105.

A user, such as a clinician or patient 105, may interact with a user interface of an external programmer 150 to program IMD 110. Programming of IMD 110 may refer generally to the generation and transfer of commands, programs, or other information to control the operation of IMD 110. In this manner, IMD 110 may receive the transferred commands and programs from external programmer 150 to control stimulation, such as electrical stimulation therapy (e.g., informed pulses) and/or control stimulation (e.g., control pulses). For example, external programmer 150 may transmit therapy stimulation programs, ECAP test stimulation programs, stimulation parameter adjustments, therapy stimulation program selections, ECAP test program selections, user input, or other information to control the operation of IMD 110, e.g., by wireless telemetry or wired connection.

In some cases, external programmer 150 may be characterized as a physician or clinician programmer if it is primarily intended for use by a physician or clinician. In other cases, external programmer 150 may be characterized as a patient programmer if it is primarily intended for use by a patient. A patient programmer may be generally accessible to patient 105 and, in many cases, may be a portable device that may accompany patient 105 throughout the patient's daily routine. For example, a patient programmer may receive input from patient 105 when the patient wishes to terminate or change electrical stimulation therapy, or when a patient perceives stimulation being delivered. In general, a physician or clinician programmer may support selection and generation of programs by a clinician for use by IMD 110, whereas a patient programmer may support adjustment and selection of such programs by a patient during ordinary use. In other examples, external programmer 150 may include, or be part of, an external charging device that recharges a power source of IMD 110. In this manner, a user may program and charge IMD 110 using one device, or multiple devices.

As described herein, information may be transmitted between external programmer 150 and IMD 110. Therefore, IMD 110 and external programmer 150 may communicate via wireless communication using any techniques known in the art. Examples of communication techniques may include, for example, radiofrequency (RF) telemetry and inductive coupling, but other techniques are also contemplated. In some examples, external programmer 150 includes a communication head that may be placed proximate to the patient's body near the IMD 110 implant site to improve the quality or security of communication between IMD 110 and external programmer 150. Communication between external programmer 150 and IMD 110 may occur during power transmission or separate from power transmission.

In some examples, IMD 110, in response to commands from external programmer 150, delivers electrical stimulation therapy according to a plurality of therapy stimulation programs to a target tissue site of the spinal cord 120 of patient 105 via electrodes (not depicted) on leads 130. In some examples, IMD 110 modifies therapy stimulation programs as therapy needs of patient 105 evolve over time. For example, the modification of the therapy stimulation programs may cause the adjustment of at least one parameter of the plurality of informed pulses. When patient 105 receives the same therapy for an extended period, the efficacy of the therapy may be reduced. In some cases, parameters of the plurality of informed pulses may be automatically updated.

In addition to, or instead of, the ECAPs-related techniques described above for sensing the effects of electrical stimulation therapy and updating parameters, this disclosure further describes techniques for sensing and analyzing local field potentials (LFPs) for determining the efficacy of electrical stimulation therapy, for updating parameters for electrical stimulation therapy, and/or for detecting changes in physiological or pain states of a patient.

As described above, one biopotential that has been used for closed-loop control is the ECAP. However, other electrical signals are also present in the spine, such as LFPs. Spinal LFPs may be alternately described as electrospinograms. In general, LFPs are rhythmic oscillations that may be detectable from the spine and related structures in the epidural space, such as DRGs, dorsal roots/rootlets, the dorsal root entry zone, and ventral roots/rootlets. Some anatomical locations may be better suited to LFP detection than others. The DRG is such an example, as recording electrodes may be located proximate neural structures being assessed (e.g., immediately proximate neural structures). The characteristics of LFP's may be indicative of various disease or neurophysiologic conditions, and the LFPs may be influenced via neuromodulation. LFPs are generally classified by measuring the power of the LFP in one or more spectral bands. In some instances, LFPs may be sensed at one location to better inform, in part, neuromodulation therapy delivered at another location.

In some examples, IMD 110 may be configured to sense LFPs from electrodes on leads 130 placed in epidural space near the spine. In some examples, the sensing electrodes may be placed near one or more DRGs, dorsal roots/rootlets, dorsal root entry zones, and ventral roots/rootlets. In general, IMD 110, external programmer 150, or another computing device may be configured analyze the sensed LFPs and determine a change in a physiological state or pain state of the patient. IMD 110 and/or external programmer 150 may be configured to use such analysis to update and/or suggest parameters for electrical stimulation therapy. For example, IMD 110 and/or external programmer 150 may change pulse width and/or amplitude of neuromodulation.

The techniques of this disclosure may further include analyzing LFPs in a frequency domain by applying a wavelet transform or another transform to sensed LFP signals. In addition, the techniques of this disclosure may include waveform processing techniques that remove electrocardiogram (ECG) and other confounding signals from the sensed LFPs so that more accurate analysis of the sensed LFPs may be performed.

As described above, ECAP analysis is a temporal analysis based on response from a stimulus. An ECAP measurement is a time-bounded event. However, an LFP is generally always present, though the amplitude of LFPs can be influenced by stimulation. ECAP and LFP detection and analysis are generally different. As such, LFP sensing may include placing leads in atypical target areas. That is, the electrodes of leads 130, and even leads 130 themselves, do not need to be placed proximally to a particular area where pain is thought to arise. In addition, IMD 110 and/or external programmer 150 may not need to analyze LFPs in any specific time window to determine a change in physiological or pain state. LFPs are present with or without stimulation. IMD 110 may be configured to measure voltage differences between two electrodes to sense LFPs. In general, intrinsic LFPs (e.g., LFPs present without stimulation) have a relatively low amplitude.

IMD 110 and/or external programmer 150 may use sensed LFPs to assess the types of neural fibers activated, the extent of neural activation, and the degree of hyper- or hypo-polarization. In general, IMD 110 and/or external programmer 150 may use the detection of LFPs as markers for conditions or physiological states that cause pain. IMD 110 and/or external programmer 150 may then use the detected changes in physiological or pain states to suggest treatments, update treatment parameters (e.g., update parameters for electrical stimulation therapy), and/or output sensed LFPs for further study.

Physical problems with the spinal cord or nervous system that cause spurious pain signals may be detectable in LFPs. Further, LFPs may be indicative of a wide range of physiologic or biochemical processes. LFPs may be indicative of slower (holistic) changes in neurological processes. Some of these may include: degree and extent of pain (neuralgia), degree of over/under sensitivity to pain (hyper- or hypoalgesia), susceptibility of a neural target to transmit or receive information, either electrically or biochemically, susceptibility of a neural target to respond therapeutically to electrical stimulation, either proximal to the location being sensed or at a distant location, or biochemical state (e.g., the up/down regulation of pain-relevant genomes, availability and susceptibility to release of signaling chemicals such as acetylcholine, Substance P, glutamate, dopamine, GABA, histamine, norepinephrine, serotonin, and glycine).

In one or more techniques of this disclosure, IMD 110 and/or external programmer 150 may be configured to analyze a sensed LFP waveform to determine LFP characteristics that are indicative of appropriate neuromodulation to the neural target (e.g., in the spine). The LFP characteristics may also indicate if the neuromodulation is not appropriate; for instance, the neuromodulation is no longer delivered to the correct anatomical location because of lead migration. IMD 110 and/or external programmer 150 may determine the LFP characteristics empirically or by a machine learning process whereby neuromodulation parameters are varied until the desired response (such as hyperalgesia suppression) is obtained. IMD 110 and/or external programmer 150 may then capture the LFP for use as a template from that point forward. Once the LFP template is obtained, IMD 110 and/or external programmer 150 may be configured to vary electrical stimulation parameters to maintain the desired response.

Example characteristics of an LFP waveform include a voltage amplitude in one or more spectral bands and/or power in one or more spectral bands. Other example characteristics may include comparisons, such as ratios, of power and/or voltage between two different spectral bands. In other examples, IMD 110 and/or external programmer 150 may be configured to measure single spike potentials in an LFP waveform (e.g., LFP signal). Spontaneous activity may occur during pain events. IMD 110 and/or external programmer 150 may be configured to measure LFPs from a dorsal root ganglion near the spinal cord over time to detect pain events. For example, IMD 110 and/or external programmer 150 may be configured to measure over a period of time (e.g., measure percent increase to detect a state change). Measuring over time does not relate to spike recording, but rather a wider measurement.

In addition to analyzing LFPs to detect changes in patient state and/or to update electrical stimulation therapy parameters, IMD 110 and/or external programmer 150 may be configured to use other sensed or input information to make therapy decisions. For example, characteristics of LFPs in addition to one or more of ECAP data, cardiac data (e.g., heart rate), respiratory data (e.g., respiration rate), and accelerometry data may be used to determine changes in patient state and/or to update electrical stimulation therapy parameters, e.g., based on the determined patient state or patient state change. For example, heart rate and respiratory rate could both feed into a multiparameter composite marker for pain. Cardiac signals can also be used to derive heart rate variability (HRV) that has been shown to have some association with pain (low HRV being a marker of elevated sympathetic tone and hence worse pain status).

LFPs may be considered a type of neural noise measurements, which may be indicative of spontaneous activity. Pain is associated with an increase in spontaneous activity of pain-carrying primary fibers in the periphery as well as cells in a DRG and/or a dorsal root. When recorded with macro-electrodes, the spontaneous activity can show up as an increase in noise levels, as well as possibly a peak in particular frequency.

IMD 110 may be configured to perform recordings of LFP signals in a position that facilitates the measurement (e.g., patient 105 is supine). Patient 105 can be automatically initiated into measurement and asked to lie down. In another example, the position can be detected from an accelerometer. Relatively long measurements may be taken and averaged, in some examples. In addition, measurements may be taken when the spinal cord is not moving. This can be determined by the ECAPs remaining relatively constant.

However, these measurements may be corrupted by ECG signals and other confounding signals. Consequently, IMD 110 and/or external programmer 150 may be configured to remove ECG signals and other confounding signals from the sensed LFP waveforms. For example, IMD 110 and/or external programmer 150 may identify ECG peaks and subtract them from the LFP waveform. The waveform that includes the LFP may also include an ECG waveform comprising a QRS complex and a T wave.

In some examples, one or more dorsal roots may intersect with the spinal cord 120 of the patient 105 at one or more locations along the spinal cord 120. These dorsal roots may represent bundles of neurons that carry signals between peripheral nerves and the spinal cord 120. For example, one or more pain signals may travel from a peripheral nerve to a DRG and/or a dorsal root. The dorsal root may carry the pain signal to a dorsal horn of the spinal cord 120. IMD 110 may deliver, via electrodes of leads 130, electrical stimulation to the dorsal root or proximate to the dorsal root in order to mitigate or completely eliminate pain that the patient 105 experiences as a result of the pain signals travelling from the dorsal root to the spinal cord 120. A proximity of the electrodes of leads 130 to the dorsal root carrying pain signals to the spinal cord 120 may determine an efficacy of electrical stimulation delivered via the leads 130. For example, when a lead (e.g., lead 130A) is placed such that electrodes of lead 130A are proximate to the dorsal root that is carrying pain signals, the IMD 110 may be configured to deliver electrical stimulation while consuming less power as compared with systems including leads that are not as proximate to the dorsal root.

One or more techniques described herein may include a system for informing a clinician as to the proximity of one or more lead electrodes to a dorsal root that is carrying pain signals to the spinal cord 120. The system may inform the clinician as to the proximity of the electrodes to a dorsal root carrying a pain signal so that the clinician may place the lead proximate to the dorsal root carrying the pain signal during an implant of the leads 130. This feedback system may allow the clinician to place the leads 130 more effectively as compared with systems that do not inform the clinician as to the proximity of electrodes to the dorsal root based on sensed bioelectric signals. This proximity indication system may be used during lead implantation and/or for selection of already implanted electrodes for sensing and/or stimulation.

In some examples, a lead of leads 130 (e.g., lead 130A) may be placed in a lateral region of the epidural space. Consequently, lead 130A may be placed proximate to one or more locations at which a dorsal root enters the spinal cord 120. IMD 110 may detect a bioelectric signal of the patient 105. In some examples, the bioelectric signal represents an LFP corresponding to one or more signals (e.g., pain signals) traveling through a dorsal root. For example, an amplitude of an LFP detected by IMD 110 may be indicative of a proximity of one or more electrodes of lead 130A to a dorsal root that is carrying pain signals to spinal cord 120. That is, the detected LFP may be indicative of one or more characteristics of the pain signal travelling through the dorsal root. IMD 110 may be configured to analyze the LFP to determine a proximity of each electrode of one or more electrodes on lead 130A to the dorsal root of the patient 105. The techniques described are not limited to analyzing LFPs. IMD 110 may also analyze one or more detected ECAPs to determine a proximity of one or more electrodes to the dorsal root. The techniques described are not limited to determining a proximity of each electrode of the one or more electrodes to a dorsal root carrying a pain signal. IMD 110 may determine a proximity of each electrode of the one or more electrodes to a dorsal root carrying a bioelectric signal that is not a pain signal (e.g., a non-pain signals) instead of, or in addition to, the pain signal.

The techniques described herein may provide clinicians with ways to target placement of medical leads during an implant procedure. The techniques described herein may provide an alternative to using dermatome maps for lead placement. By determining the location of electrodes relative to a dorsal root and outputting the electrode locations in real time, the system may decrease an amount of time for completing the implant procedure as compared with systems that do not output a location of the electrode relative to the dorsal root in real time. It may be beneficial to place a lead in a lateral region of the epidural space, so that the IMD 110 may use detected ECAPs and/or LFPs to sense pain signals entering the dorsal horn. Since the lateral region of the epidural space is proximate to one or more locations where the dorsal roots enter the spinal cord 120, the techniques described herein may provide information corresponding to a location of a pain signal origination in the central nervous system, thus providing information that may assist in targeting physiological placement of electrodes of IMD 110. This information may allow a physician to place the lead for optimal stimulation. In other examples, the techniques described herein may provide a clinician with an indication of the location of already implanted electrodes such that the clinician, or system, can determine electrode configurations for stimulation delivery.

In some examples, a lead located in the lateral area of the epidural space may be differentiated from a lead located more centrally in the epidural space using ECAP signals. For example, the lateral area closer to the dorsal horn may sense ECAPs with a slower, or broader, ECAP signal because the sensed signal may include C-fibers which are slower to propagate signal that other fibers. When moving the lead, ECAPs with a broader peaks or greater latency may be indicative of placement more laterally in the epidural space as compared to a lead located more central in the epidural space.

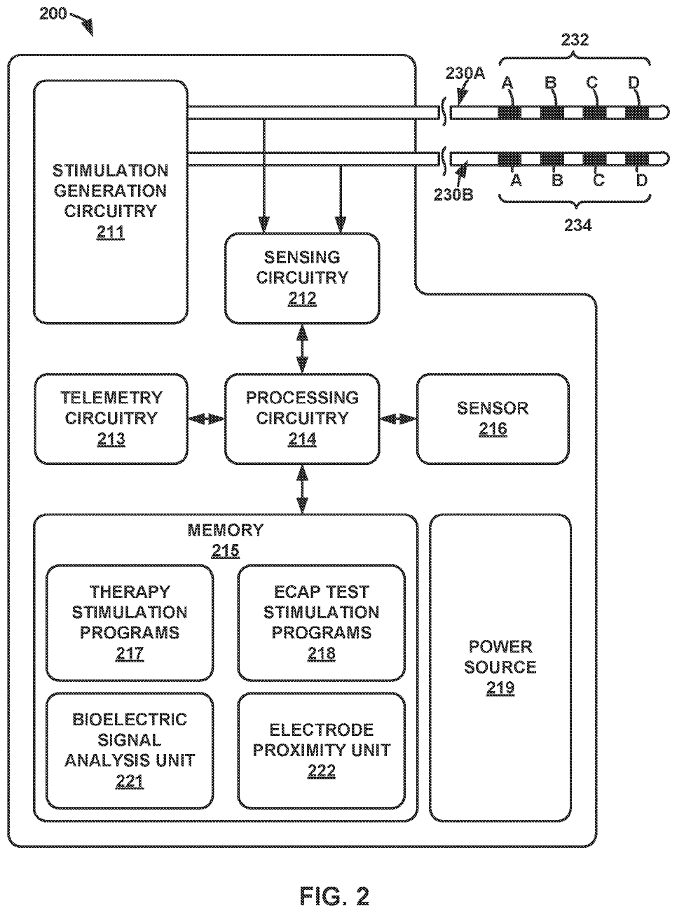

FIG. 2 is a block diagram of IMD 200, in accordance with one or more techniques of this disclosure. IMD 200 may be an example of IMD 110 of FIG. 1. In the example shown in FIG. 2, IMD 200 includes processing circuitry 214, memory 215, stimulation generation circuitry 211, sensing circuitry 212, telemetry circuitry 213, sensor 216, and power source 219. Each of these circuits may be or include programmable or fixed function circuitry configured to perform the functions attributed to respective circuitry. For example, processing circuitry 214 may include fixed-function or programmable circuitry, stimulation generation circuitry 211 may include circuitry configured to generate stimulation signals such as pulses or continuous waveforms on one or more channels, sensing circuitry 212 may include sensing circuitry for sensing signals, and telemetry circuitry 213 may include telemetry circuitry for transmission and reception of signals. Memory 215 may store computer-readable instructions that, when executed by processing circuitry 214, cause IMD 200 to perform various functions. Memory 215 may be a storage device or other non-transitory medium.

In the example shown in FIG. 2, memory 215 stores therapy stimulation programs 217 and ECAP test stimulation programs 218 in separate memories within memory 215 or separate areas within memory 215. Memory 215 also stores bioelectric signal analysis unit 221 and electrode proximity unit 222. Each stored therapy stimulation program 217 defines values for a set of electrical stimulation parameters (e.g., a parameter set or set of parameter values), such as a stimulation electrode combination, electrode polarity, current or voltage amplitude, pulse width, pulse rate, and pulse shape. Each stored ECAP test stimulation programs 218 defines values for a set of electrical stimulation parameters (e.g., a control stimulation parameter set), such as a stimulation electrode combination, electrode polarity, current or voltage amplitude, pulse width, pulse rate, and pulse shape. ECAP test stimulation programs 218 may also have additional information such as instructions regarding when to deliver control pulses based on the pulse width and/or frequency of the informed pulses defined in therapy stimulation programs 217.

Accordingly, in some examples, stimulation generation circuitry 211 generates electrical stimulation signals in accordance with the electrical stimulation parameters noted above. Other ranges of parameter values may also be useful and may depend on the target stimulation site within patient 105. While stimulation pulses are described, stimulation signals may be of any form, such as continuous-time signals (e.g., sine waves) or the like.

Processing circuitry 214 may include any one or more of a microprocessor, a controller, a digital signal processor (DSP), an application specific integrated circuit (ASIC), a field-programmable gate array (FPGA), discrete logic circuitry, or any other processing circuitry configured to provide the functions attributed to processing circuitry 214 herein may be embodied as firmware, hardware, software or any combination thereof. Processing circuitry 214 controls stimulation generation circuitry 211 to generate stimulation signals according to therapy stimulation programs 217 and ECAP test stimulation programs 218 stored in memory 215 to apply stimulation parameter values specified by one or more of programs, such as amplitude, pulse width, pulse rate, and pulse shape of each of the stimulation signals.

In the example shown in FIG. 2, the set of electrodes 232 includes electrodes 232A, 232B, 232C, and 232D, and the set of electrodes 234 includes electrodes 234A, 234B, 234C, and 234D. In other examples, a single lead may include all eight electrodes 232 and 234 along a single axial length of the lead. Processing circuitry 214 also controls stimulation generation circuitry 211 to generate and apply the stimulation signals to selected combinations of electrodes 232, 234. In some examples, stimulation generation circuitry 211 includes a switch circuit that may couple stimulation signals to selected conductors within leads 230, which, in turn, deliver the stimulation signals across selected electrodes 232, 234. Such a switch circuit may be a switch array, switch matrix, multiplexer, or any other type of switching circuit configured to selectively couple stimulation energy to selected electrodes 232, 234 and to selectively sense bioelectrical neural signals of a spinal cord of the patient (not shown in FIG. 2) with selected electrodes 232, 234.

In other examples, however, stimulation generation circuitry 211 does not include a switch circuit. In these examples, stimulation generation circuitry 211 comprises a plurality of pairs of voltage sources, current sources, voltage sinks, or current sinks connected to each of electrodes 232, 234 such that each pair of electrodes has a unique signal circuit. In other words, in these examples, each of electrodes 232, 234 is independently controlled via its own signal circuit (e.g., via a combination of a regulated voltage source and sink or regulated current source and sink), as opposed to switching signals between electrodes 232, 234.

Electrodes 232, 234 on respective leads 230 may be constructed of a variety of different designs. For example, one or both of leads 230 may include one or more electrodes at each longitudinal location along the length of the lead, such as one electrode at different perimeter locations around the perimeter of the lead at each of the locations A, B, C, and D. In one example, the electrodes may be electrically coupled to stimulation generation circuitry 211, via respective wires that are straight or coiled within the housing of the lead and run to a connector at the proximal end of the lead. In another example, each of the electrodes of the lead may be electrodes deposited on a thin film. The thin film may include an electrically conductive trace for each electrode that runs the length of the thin film to a proximal end connector. The thin film may then be wrapped (e.g., a helical wrap) around an internal member to form the lead 230. These and other constructions may be used to create a lead with a complex electrode geometry.

Although sensing circuitry 212 is incorporated into a common housing with stimulation generation circuitry 211 and processing circuitry 214 in FIG. 2, in other examples, sensing circuitry 212 may be in a separate housing from IMD 200 and may communicate with processing circuitry 214 via wired or wireless communication techniques.

In some examples, one or more of electrodes 232 and 234 may be suitable for sensing the ECAPs. For instance, electrodes 232 and 234 may sense the voltage amplitude of a portion of the ECAP signals, where the sensed voltage amplitude is a characteristic the ECAP signal.

Sensor 216 may include one or more sensing elements that sense values of a respective patient parameter. As described, electrodes 232 and 234 may be the electrodes that sense the parameter value of the ECAP. Sensor 216 may include one or more accelerometers, optical sensors, chemical sensors, temperature sensors, pressure sensors, or any other types of sensors. Sensor 216 may output patient parameter values that may be used as feedback to control delivery of therapy. For example, sensor 216 may indicate patient activity, and processing circuitry 214 may increase the frequency of control pulses and ECAP sensing in response to detecting increased patient activity. In one example, processing circuitry 214 may initiate control pulses and corresponding ECAP sensing in response to a signal from sensor 216 indicating that patient activity has exceeded an activity threshold. Conversely, processing circuitry 214 may decrease the frequency of control pulses and ECAP sensing in response to detecting decreased patient activity. For example, in response to sensor 216 no longer indicating that the sensed patient activity exceeds a threshold, processing circuitry 214 may suspend or stop delivery of control pulses and ECAP sensing. In this manner, processing circuitry 214 may dynamically deliver control pulses and sense ECAP signals based on patient activity to reduce power consumption of the system when the electrode-to-neuron distance is not likely to change and increase system response to ECAP changes when electrode-to-neuron distance is likely to change. IMD 200 may include additional sensors within the housing of IMD 200 and/or coupled via one of leads 130 or other leads. In addition, IMD 200 may receive sensor signals wirelessly from remote sensors via telemetry circuitry 213, for example. In some examples, one or more of these remote sensors may be external to patient (e.g., carried on the external surface of the skin, attached to clothing, or otherwise positioned external to the patient). In some examples, signals from sensor 216 may indicate a position or body state (e.g., sleeping, awake, sitting, standing, or the like), and processing circuitry 214 may select target ECAP characteristic values according to the indicated position or body state.

Telemetry circuitry 213 is configured to provide wireless communication between IMD 200 and an external programmer (not shown in FIG. 2) or another computing device under the control of processing circuitry 214. Processing circuitry 214 of IMD 200 may receive, as updates to programs, values for various stimulation parameters such as amplitude and electrode combination, from the external programmer via telemetry circuitry 213. Updates to the therapy stimulation programs 217 and ECAP test stimulation programs 218 may be stored within memory 215. Telemetry circuitry 213 in IMD 200, as well as telemetry circuits in other devices and systems described herein, such as the external programmer, may accomplish communication by radiofrequency (RF) communication techniques. In addition, telemetry circuitry 213 may communicate with an external medical device programmer (not shown in FIG. 2) via proximal inductive interaction of IMD 200 with the external programmer. The external programmer may be one example of external programmer 150 of FIG. 1. Accordingly, telemetry circuitry 213 may send information to the external programmer on a continuous basis, at periodic intervals, or upon request from IMD 110 or the external programmer.

Power source 219 delivers operating power to various components of IMD 200. Power source 219 may include a rechargeable or non-rechargeable battery and a power generation circuit to produce the operating power. Recharging may be accomplished through proximal inductive interaction between an external charger and an inductive charging coil within IMD 200. In other examples, traditional primary cell batteries may be used.

In some examples, stimulation generation circuitry 211 of IMD 200 receives, via telemetry circuitry 213, instructions to deliver electrical stimulation therapy according to therapy stimulation programs 217 to a target tissue site of the spinal cord of the patient via a plurality of electrode combinations of electrodes 232, 234 of leads 230 and/or a housing of IMD 200. Stimulation generation circuitry 211 may receive, via telemetry circuitry 213, user instructions to deliver control stimulation to the patient according to ECAP test stimulation programs 218. Each pulse of a plurality of control pulses may elicit an ECAP that is sensed by sensing circuitry 212 via some of electrodes 232 and 234. ECAP test stimulation programs 218 may instruct stimulation generation circuitry 211 to deliver a plurality of control pulses interleaved with at least some of the plurality of informed pulses. Processing circuitry 214 may receive, via an electrical signal sensed by sensing circuitry 212, information indicative of an ECAP signal (e.g., a numerical value indicating a characteristic of the ECAP in electrical units such as voltage or power) produced in response to the control stimulation. Therapy stimulation programs 217 may be updated according to the ECAPs recorded at sensing circuitry 212 according to the following techniques.

In one example, the plurality of informed pulses each have a pulse width of greater than approximately 100 □s and less than approximately 2000 □s (i.e., 2 milliseconds). In some examples, the informed pulse width is greater than approximately 200 □s and less than approximately 800 □s. In another example, the informed pulse width is greater than approximately 300 □s and less than approximately 500 □s. In one example, informed pulses have a pulse width of approximately 450 □s and a pulse frequency of approximately 60 Hertz. Amplitude (current and/or voltage) for the informed pulses may be between approximately 0.5 mA (or volts) and approximately 10 mA (or volts), although amplitude may be lower or greater in other examples. In some examples, the system may deliver informed pulses from two or more stimulation programs such that the informed pulses from one stimulation program have at least one different parameter value than the informed pulses from another stimulation program.

Each control pulse of the plurality of control pulses may have a pulse width of less than approximately 300 □s. In one example, each control pulse of the plurality of control pulses may be a bi-phasic pulse with a positive phase having a width of approximately 100 □s, a negative phase having a width of approximately 100 □s, and an interphase interval having a width of approximately 30 □s. In some examples, the positive phase and negative phase may each be 90 □s or 120 □s in other examples. In other examples, the control pulses may each have a pulse width of approximately 60 □s or smaller. Due to the relatively long pulse widths of the plurality of informed pulses, sensing circuitry 212 may be incapable of adequately recording an ECAP signals elicited from an informed pulse because the informed pulse itself will occur during the ECAP signal and obscure the ECAP signal. However, stimulation pulses with pulse widths less than approximately 300 microseconds, such as the plurality of control pulses, may be suited to elicit an ECAP which can be sensed after the control pulse is completed at sensing circuitry 212 via two or more of electrodes 232 and 234. In some examples, the control pulses may be non-therapeutic pulses in that the control pulses do not contribute to therapy for the patient. In other examples, the control pulses may fully provide or partially contribute to the therapy received by the patient by reducing or eliminating symptoms and/or a condition of the patient.

Control pulses delivered for the purpose of eliciting detectable ECAP signals may have a current amplitude between approximately 6 mA and 12 mA in some examples, but higher or lower amplitudes may be used in other examples. The frequency of the control pulses may be between approximately 50 Hertz and 400 Hertz in some examples, which may match the predetermined pulse frequency of the informed pulses when one control pulse is delivered for each therapeutic pulse. The predetermined pulse frequency may be a single frequency or a varied frequency over time (e.g., the interpulse interval may change over time according to predetermined pattern, formula, or schedule). In some examples, the system may change the predetermined pulse frequency based on patient input or a sensed parameter such as patient posture or activity. Such a relationship may be present when the control pulses are fully interleaved (e.g., alternating) with the informed pulses. However, the frequency of the control pulses may be delivered at a higher frequency than then informed pulses when two or more control pulses are delivered between consecutive informed pulses. In other examples, the frequency of the control pulses may be delivered at a lower frequency than the informed pulses when at least some informed pulses are delivered without a control pulse delivered between them. The frequency of the control pulses may be delivered at a frequency that varies over time if the system is configured to adjust control pulse delivery, and the resulting ECAP sensing, based on other factors such as patient activity.

In one example, the predetermined pulse frequency of the plurality of informed pulses may be less than approximately 400 Hertz. In some examples, the predetermined pulse frequency of the plurality of informed pulses may be between approximately 50 Hertz and 70 Hertz. In one example, the predetermined pulse frequency of the plurality of informed pulses may be approximately 60 Hertz. However, the informed pulses may have frequencies greater than 400 Hertz or less than 50 Hertz in other examples. In some examples, the predetermined pulse frequency of the informed pulses may be a single frequency or a frequency that varies over time. In addition, the informed pulses may be delivered in bursts of pulses, with interburst frequencies of the pulses being low enough such that a control pulse and sensed ECAP can still fit within the window between consecutive informed pulses delivered within the burst of informed pulses.

Since each informed pulse of the plurality of informed pulses may be sensed as an artifact that covers, or obscures, the sensing of at least one ECAP, the plurality of control pulses may be delivered to the patient during a plurality of time events. For example, a time event (e.g., a window) of the plurality of time events may be a time (e.g., a window) between consecutive informed pulses of the plurality of informed pulses at the predetermined pulse frequency. One or more control pulses of the plurality of control pulses may be delivered to the patient during each time event. Consequently, the control pulses may be interleaved with at least some of the informed pulses such that the plurality of control pulses are delivered to the patient while informed pulses are not delivered. In one example, an ECAP elicited from to a control pulse delivered during a time event may be recorded by sensing circuitry 212 during the same time event. In another example, two or more ECAPs responsive to two or more respective control pulses delivered during a time event may be recorded by sensing circuitry 212 during the same time event.

In some examples, therapy stimulation programs 217 may be updated according to a plurality of ECAPs received in response to the plurality of control pulses delivered to the patient according to ECAP test stimulation programs 218. For instance, processing circuitry 214 may update therapy stimulation programs 217 in real time by comparing one or more characteristics of ECAPs sensed by sensing circuitry 212 with target ECAP characteristics stored in memory 215. For example, processing circuitry 214 is configured to determine the amplitude of each ECAP signal received at sensing circuitry 212, and processing circuitry 214 is further configured to determine the representative amplitude of at least one respective ECAP signal and compare the representative amplitude of a series of ECAP signals to a target ECAP adjustment window. Target ECAP adjustment window may thus be a range of amplitudes deviating from target ECAP amplitude. For instance, the target ECAP adjustment window may span from a lower-bound amplitude value (e.g., the target ECAP amplitude minus the variance) to an upper-bound amplitude value (e.g., the target ECAP amplitude plus the variance). Generally, the lower-bound amplitude value is less than the target ECAP amplitude, and the upper-bound amplitude value is greater than target ECAP amplitude.

If the representative amplitude of the at least one respective ECAP signal (e.g., an amplitude of a single ECAP signal or an average of two or more ECAP amplitudes) is greater than the upper-bound amplitude value, processing circuitry 214 may adjust one or more of therapy stimulation programs 217 and ECAP test stimulation programs 218 to decrease the amplitude of informed pulses and control pulses following the at least one respective ECAP. The amplitude of informed pulses and control pulses may be decreased by different predetermined steps or different predetermined percentages. Additionally, if the representative amplitude of the at least one respective ECAP is less than the lower-bound amplitude value, processing circuitry 214 may adjust therapy stimulation programs 217 and ECAP test stimulation programs 218, and the programs 217 and 218 may instruct stimulation generation circuitry 211 to increase the amplitude of informed pulses and control pulses following the at least one respective ECAP. Moreover, if the representative amplitude of the at least one respective ECAP is greater than the lower-bound amplitude value and less than the upper-bound amplitude value, processing circuitry 214 may not change programs 217 and 218, and stimulation generation circuitry 211 may maintain the amplitude of the informed pulses following the at least one respective ECAP. In one example, adjusting the programs 217 and 218 may include changing one or more parameters of the plurality of informed pulses and the plurality of control pulses. In one example, the at least one respective ECAP may include a series of four consecutive ECAPs.

Processing circuitry 214, in one example, may change the amplitude of the informed pulses and the control pulses following the at least one respective ECAP inversely proportional to the difference between target ECAP amplitude and the representative amplitude of the at least one respective ECAP. For instance, if the representative amplitude of the at least one respective ECAP is 20% lower than target ECAP amplitude, then processing circuitry 214 may update therapy programs 217 and 218 such that the amplitude of informed pulses and the control pulses is increased by 20%. In one example, the representative amplitude may be the mean amplitude of two or more respective ECAP signals sensed by sensing circuitry 212. In other examples, the representative amplitude may be the median amplitude of two or more respective ECAP signal, or a rolling average of two or more respective ECAP signals.

In another example, processing circuitry 214 may determine the amplitude of a respective ECAP signal sensed by sensing circuitry 212. In response to a comparison between the amplitude of the respective ECAP signal and a target ECAP amplitude, processing circuitry 214 may determine a percentage difference between the amplitude of the respective ECAP signal and target ECAP amplitude. Consequently, processing circuitry 214 may adjust the amplitude of subsequent informed pulses to be inversely proportional to the percentage difference between the amplitude of the respective ECAP and target ECAP amplitude.

In other examples, processing circuitry 214 may use the representative amplitude of the at least one respective ECAP to change other parameters of informed pulses to be delivered, such as pulse width, pulse frequency, and pulse shape. All of these parameters may contribute to the intensity of the informed pulses, and changing one or more of these parameter values may effectively adjust the informed pulse intensity to compensate for the changed distance between the stimulation electrodes and the nerves indicated by the representative amplitude of the ECAP signals.

In some examples, leads 230 may be linear 8-electrode leads (not pictured); sensing and stimulation delivery may each be performed using a different set of electrodes. In a linear 8-electrode lead, each electrode may be numbered consecutively from 0 through 7. For instance, a control pulse may be generated using electrode 1 as a cathode and electrodes 0 and 2 as anodes (e.g., a guarded cathode), and a respective ECAP signal may be sensed using electrodes 6 and 7, which are located on the opposite end of the electrode array. This strategy may minimize the interference of the stimulation pulse with the sensing of the respective ECAP. Other electrode combinations may be implemented, and the electrode combinations may be changed using the patient programmer via telemetry circuitry 213. For example, stimulation electrodes and sensing electrodes may be positioned closer together. Shorter pulse widths for the control pulses may allow the sensing electrodes to be closer to the stimulation electrodes.

In some examples, IMD 200 may be configured to detect, using sensing circuitry 212, one or more bioelectric signals corresponding to pain signals travelling through the nervous system of patient 105. These bioelectric signals may include any one or combination of LFPs, ECAPs, and other kinds of signals. In some examples, sensing circuitry 212 may receive the one or more bioelectric signals from any one or combination of electrodes 232, 234 during an implant of leads 230. That is, the detected bioelectric signals may include information indicative of a position of lead 230A and/or lead 230B during an implant of leads 230.

In some examples, a clinician may implant leads 230 in an epidural space of patient 105. The epidural space represents a gap in a central area of the vertebrae, the gap extending along at least a portion of the spinal cord 120. In some examples, the epidural space may represent a space between a posterior portion of a vertebra of the patient 105 and the spinal cord 120 of the patient. It may be beneficial for a clinician to implant leads 230 in the epidural space. Because the epidural space is proximate to the spinal cord 120, leads placed within the epidural space may stimulate the spinal cord 120 and/or other nerve tissue proximate to the spinal cord 120 The epidural space of the patient may include a central region and one or more lateral regions. The central region of the epidural space may include a center point of a cross-section of the epidural space. The lateral regions of the epidural place may be laterally displaced from the center point of the cross-section of the epidural space. In some examples, the epidural space may include a first lateral region, a second lateral region, and a central region. The lateral regions of the epidural space, in some cases, may be narrower than the central region of the epidural space. Consequently, the central region of the epidural space may include a greater volume of fat as compared with each lateral region of the epidural space.

The spinal cord 120 may include the dorsal horn. Dorsal horn neurons may carry signals along the spinal cord 120 to and from a brain of patient 105. Dorsal roots may enter the spinal cord 120 of the patient 105 at one or more locations along a length of the spinal cord 120. Dorsal roots may serve as a bridge between the dorsal horn and one or more peripheral nerves of the patient. In some examples, patient 105 may experience pain when the dorsal horn carries pain signals to the patient's brain. In some cases, these pain signals may enter the dorsal horn via one or dorsal roots. In some examples, a lateral region of the epidural space may be proximate to a point at which a dorsal root enters the spinal cord 120 and connects with the dorsal horn. In some examples, one or more of leads 130 may be placed in a lateral region of the epidural space of the patient 105 so that the one or more of leads 130 is proximate to one or more locations in which dorsal roots enter the spinal cord 120.

For example, processing circuitry 214 may be configured to determine, based on one or more bioelectric signals sensed by sensing circuitry 212 via one of leads 230 (e.g., lead 230A), a proximity of lead 230A to a dorsal root carrying pain signals to the dorsal horn. In some examples, processing circuitry 214 may execute bioelectric signal analysis unit 221 in order to determine a proximity of each of electrodes 232 to the dorsal root carrying pain signals to the dorsal horn. For example, when a clinician is implanting lead 230A in a lateral region of the epidural space of patient 105, sensing circuitry 212 may receive the one or more bioelectric signals via electrodes 232. In some examples, the one or more bioelectric signals may include a set of bioelectric signal components, where each bioelectric signal component of the set of bioelectric signal components corresponds to a respective electrode of electrodes 232. For example, a first bioelectric signal component may correspond to electrode 232A, a second bioelectric signal component may correspond to electrode 232B, a third bioelectric signal component may correspond to electrode 232C, and a fourth bioelectric signal component may correspond to electrode 232D. Processing circuitry 214 may execute bioelectric signal analysis unit 221 in order to identify one or more characteristics of each bioelectric signal component of the set of bioelectric signal components. Additionally, in some examples, processing circuitry 214 may execute bioelectric signal analysis unit 221 in order to determine, based on the one or more characteristics of each bioelectric signal component of the set of bioelectric signal components, the proximity of each electrode of electrodes 232 to the dorsal root.

In some examples, the one or more bioelectric signals detected by sensing circuitry 212 may include LFPs. LFPs may represent bioelectric signals that are caused by the human body and not elicited by electrical stimulation pulses. Consequently, IMD 200 may sense LFPs without delivering electrical stimulation pulses to patient 105. In some examples, sensing circuitry 212 may sense one or more LFPs corresponding to a dorsal root carrying pain signals to the dorsal horn. LFPs may be stronger in areas closer to the dorsal root. For example, when sensing circuitry 212 detects a first one or more LFPs via a first one or more electrodes and detects a second one or more LFPs via a second one or more electrodes, an amplitude of the first one or more LFPs may be greater than an amplitude of the second one or more LFPs when the first one or more electrodes are closer to the dorsal root than the second one or more electrodes. Amplitude is not necessarily the only LFP parameter that changes based on proximity to a dorsal root. Pulse shape, pulse frequency, and other parameters may also change based on proximity to the dorsal root. Based on sets of LFPs corresponding to each of electrodes 232, processing circuitry 214 may execute bioelectric signal analysis unit 221 in order to determine a proximity of each of electrodes 232 to the dorsal root carrying pain signals.

In some examples, the one or more bioelectric signals detected by sensing circuitry 212 may include ECAPs. ECAPs are elicited by stimulation pulses. To elicit ECAPs, IMD 200 may deliver a sequence of stimulation pulses to patient 105 while a clinician is implanting lead 230A in a lateral region of the epidural space. Sensing circuitry 212 may detect, in response to one or more stimulation pulses of the sequence of stimulation pulses, a respective ECAP.

IMD 200 may, in some examples, deliver the sequence of stimulation pulses via lead 230A while the clinician implants the lead 230A in the patient 105, and sensing circuitry 212 may detect the ECAPs via lead 230A. For example, IMD 200 may deliver stimulation pulses and detect ECAPs using the same lead. When IMD 200 delivers the sequence of stimulation pulses to the patient 105 using lead 230A, IMD 200 may elicit ECAPs that travel through the dorsal root that is carrying pain signals to the dorsal horn. One or more characteristics of these ECAPs may correspond to a severity of the pain experienced by patient 105 as a result of the pain signals traveling through the dorsal root to the dorsal horn. For example, an amplitude of the ECAPs may increase as a level of pain experienced by the patient increases. Consequently, one or more characteristics of ECAPs elicited in a dorsal root may indicate one or more characteristics of pain signals travelling through the dorsal root.

IMD 200 may, in some examples, deliver the sequence of stimulation pulses via lead 230B while the clinician implants the lead 230A in the patient 105, and sensing circuitry 212 may detect the ECAPs via lead 230A. In some examples, lead 230B may be placed in a central region of the epidural space. That is, IMD 200 delivers stimulation at the central region of the epidural space and detects one or more ECAPS via lead 230A located in a lateral region of the epidural space. When IMD 200 delivers the sequence of stimulation pulses to the patient 105 using lead 230B, IMD 200 may elicit ECAPs in the dorsal horn that travel through the dorsal root that is carrying pain signals to the dorsal horn. One or more characteristics of these ECAPs may correspond to a severity of the pain experienced by patient 105 as a result of the pain signals traveling through the dorsal root to the dorsal horn. For example, an amplitude of the ECAPs may increase as a level of pain experienced by the patient increases. Consequently, one or more characteristics of ECAPs elicited at the dorsal horn and travelling through the a dorsal root may indicate one or more characteristics of pain signals travelling through the dorsal root.

In some examples, IMD 200 may output information indicative of the proximity of each electrode of electrodes 232 to the dorsal root. For example, processing circuitry 214 may execute electrode proximity unit 222 in order to determine, based on the one or more bioelectric signals, the proximity of electrode 232A to the dorsal root, a proximity of electrode 232B to the dorsal root, a proximity of electrode 232C to the dorsal root, and a proximity of electrode 232D to the dorsal root. IMD 200 may output, via the telemetry circuitry 213, the respective proximity of each of electrodes 232 to the dorsal root. In some examples, a user interface may display the proximity of each of electrodes 232 to the dorsal root in real time or near real-time so that a clinician can place electrodes 232 proximate to the dorsal root. The proximity information may enable the clinician to place lead 230A so that electrodes 232 are in a beneficial location to deliver stimulation while conserving power from power source 219. In another example, the proximity information may enable the clinician to select which electrodes from lead 230A and/or lead 230B are to be included in the electrode combination used for stimulation.

In one example, sensor 216 may detect a change in activity or a change in posture of the patient. Processing circuitry 214 may receive an indication from sensor 216 that the activity level or posture of the patient is changed, and processing circuitry 214 may be configured to initiate or change the delivery of the plurality of control pulses according to the ECAP test stimulation programs 218. For example, processing circuitry 214 may increase the frequency of control pulse delivery and respective ECAP sensing in response to receiving an indication that the patient activity has increased, which may indicate that the distance between electrodes and nerves will likely change. Alternatively, processing circuitry 214 may decrease the frequency of control pulse delivery and respective ECAP sensing in response to receiving an indication that the patient activity has decreased. In some examples, one or more parameters (e.g., frequency, amplitude, slew rate, pulse duration, or the like) may be adjusted (e.g., increased or decreased) in response to receiving an indication that the patient activity has changed. Processing circuitry 214 may be further configured to update therapy stimulation programs 217 and ECAP test stimulation programs 218 according to the signal received from sensor 216.

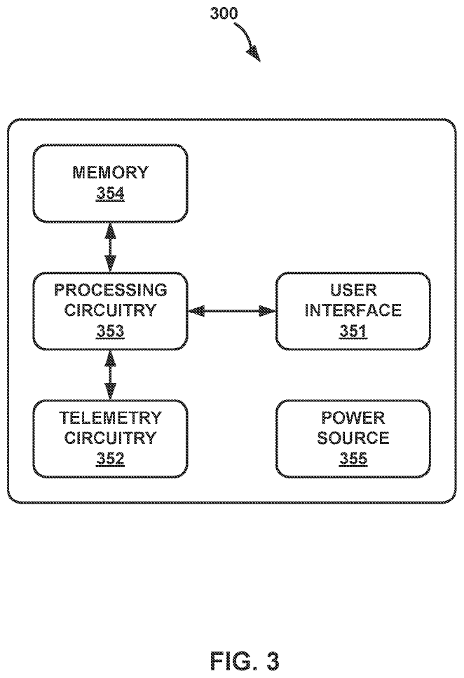

FIG. 3 is a block diagram of the example external programmer 300, in accordance with one or more techniques of this disclosure. External programmer 300 may be an example of external programmer 150 of FIG. 1. Although programmer 300 may generally be described as a hand-held device, programmer 300 may be a larger portable device or a more stationary device. In addition, in other examples, programmer 300 may be included as part of an external charging device or include the functionality of an external charging device. As illustrated in FIG. 3, programmer 300 may include a processing circuitry 353, memory 354, user interface 351, telemetry circuitry 352, and power source 355. Memory 354 may store instructions that, when executed by processing circuitry 353, cause processing circuitry 353 and external programmer 300 to provide the functionality ascribed to external programmer 300 throughout this disclosure. Each of these components, circuitry, or modules, may include electrical circuitry that is configured to perform some, or all of the functionality described herein. For example, processing circuitry 353 may include processing circuitry configured to perform the processes discussed with respect to processing circuitry 353.

In general, programmer 300 comprises any suitable arrangement of hardware, alone or in combination with software and/or firmware, to perform the techniques attributed to programmer 300, and processing circuitry 353, user interface 351, and telemetry circuitry 352 of programmer 300. In various examples, programmer 300 may include one or more processors, such as one or more microprocessors, DSPs, ASICs, FPGAs, or any other equivalent integrated or discrete logic circuitry, as well as any combinations of such components. Programmer 300 also, in various examples, may include a memory 354, such as random access memory (RAM), read only memory (ROM), programmable read only memory (PROM), erasable programmable read only memory (EPROM), electronically erasable programmable read only memory (EEPROM), flash memory, a hard disk, a CD-ROM, comprising executable instructions for causing the one or more processors to perform the actions attributed to them. Moreover, although processing circuitry 353 and telemetry circuitry 352 are described as separate modules, in some examples, processing circuitry 353 and telemetry circuitry 352 are functionally integrated. In some examples, processing circuitry 353 and telemetry circuitry 352 correspond to individual hardware units, such as ASICs, DSPs, FPGAs, or other hardware units.

Memory 354 (e.g., a storage device) may store instructions that, when executed by processing circuitry 353, cause processing circuitry 353 and programmer 300 to provide the functionality ascribed to programmer 300 throughout this disclosure. For example, memory 354 may include instructions that cause processing circuitry 353 to obtain a parameter set from memory, select a spatial electrode movement pattern, or receive a user input and send a corresponding command to IMD 110, or instructions for any other functionality. In addition, memory 354 may include a plurality of programs, where each program includes a parameter set that defines therapy stimulation or control stimulation. Memory 354 may also store data received from a medical device (e.g., IMD 110). For example, memory 354 may store ECAP related data recorded at a sensing module of the medical device, and memory 354 may also store data from one or more sensors of the medical device. Additionally, or alternatively, memory 354 may store LFP related data recorded at a sensing module of the medical device. Memory 354 may also store data indicative of a proximity of one or more electrodes to a dorsal root carrying pain signals to the dorsal horn.

User interface 351 may include a button or keypad, lights, a speaker for voice commands, a display, such as a liquid crystal (LCD), light-emitting diode (LED), or organic light-emitting diode (OLED). In some examples the display may be a touch screen. User interface 351 may be configured to display any information related to the delivery of electrical stimulation, sensing of one or more bioelectric signals, implant of one or more leads, identified patient behaviors, sensed patient parameter values, patient behavior criteria, or any other such information. User interface 351 may also receive user input via user interface 351. The input may be, for example, in the form of pressing a button on a keypad or selecting an icon from a touch screen. The input may request starting or stopping electrical stimulation, the input may request a new spatial electrode movement pattern or a change to an existing spatial electrode movement pattern, of the input may request some other change to the delivery of electrical stimulation.

Telemetry circuitry 352 may support wireless communication between the medical device and programmer 300 under the control of processing circuitry 353. Telemetry circuitry 352 may also be configured to communicate with another computing device via wireless communication techniques, or direct communication through a wired connection. In some examples, telemetry circuitry 352 provides wireless communication via an RF or proximal inductive medium. In some examples, telemetry circuitry 352 includes an antenna, which may take on a variety of forms, such as an internal or external antenna.

Examples of local wireless communication techniques that may be employed to facilitate communication between programmer 300 and IMD 110 include RF communication according to the 802.11 or Bluetooth specification sets or other standard or proprietary telemetry protocols. In this manner, other external devices may be capable of communicating with programmer 300 without needing to establish a secure wireless connection. As described herein, telemetry circuitry 352 may be configured to transmit a spatial electrode movement pattern or other stimulation parameter values to IMD 110 for delivery of electrical stimulation therapy.

In some examples, selection of parameters or therapy stimulation programs may be transmitted to the medical device for delivery to the patient. In other examples, the therapy may include medication, activities, or other instructions that the patient must perform themselves or a caregiver perform for the patient. In some examples, programmer 300 may provide visual, audible, and/or tactile notifications that indicate there are new instructions. Programmer 300 may require receiving user input acknowledging that the instructions have been completed in some examples.

According to the techniques of the disclosure, user interface 351 of external programmer 300 receives an indication from a clinician instructing a processor of the medical device to update one or more therapy stimulation programs or to update one or more ECAP test stimulation programs. Updating therapy stimulation programs and ECAP test stimulation programs may include changing one or more parameters of the stimulation pulses delivered by the medical device according to the programs, such as amplitude, pulse width, frequency, and pulse shape of the informed pulses and/or control pulses. User interface 351 may also receive instructions from the clinician commanding any electrical stimulation, including therapy stimulation and control stimulation to commence or to cease.

The architecture of programmer 300 illustrated in FIG. 3 is shown as an example. The techniques as set forth in this disclosure may be implemented in the example programmer 300 of FIG. 3, as well as other types of systems not described specifically herein. Nothing in this disclosure should be construed so as to limit the techniques of this disclosure to the example architecture illustrated by FIG. 3.

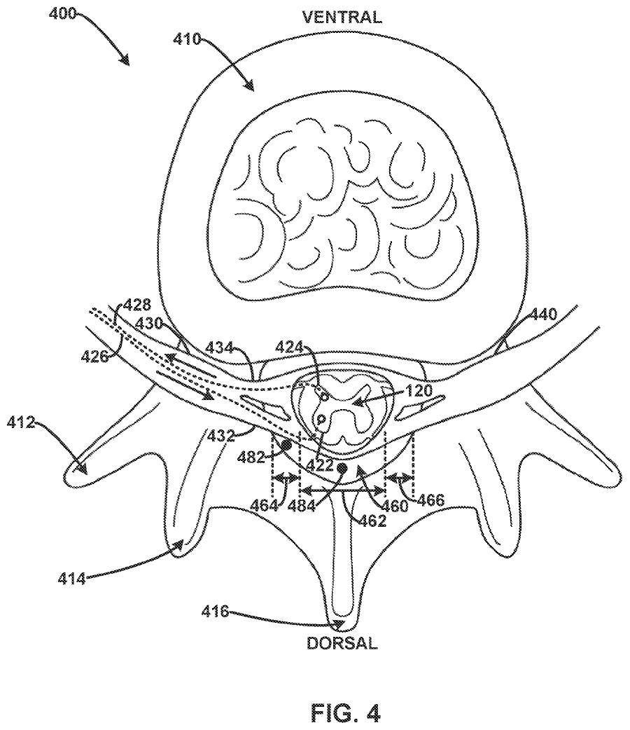

FIG. 4 is a conceptual diagram illustrating a cross-section of a spinal column 400, in accordance with one or more techniques of this disclosure. Spinal column 400 includes a vertebra including a vertebral body 410, a transverse process 412, a superior articular process 414, and a spinous process 416. Spinal column 400 also includes a spinal cord 120 including dorsal horn 422 and ventral horn 424. In some examples, spinal cord 120 extends along at least a portion of a length of the spinal column 400 to the brain of patient 105. As seen in FIG. 4, the cross-section of the spinal column 400 includes a first spinal nerve 430 and a second spinal nerve 440. The first spinal nerve 430 may include a plurality of neurons that connect with spinal cord 120. First spinal nerve 430 includes dorsal root 432 and ventral root 434. At least some neurons of spinal nerve 430 may pass through dorsal root 432, and at least some neurons of spinal nerve 430 may pass through ventral root 434. For example, one or more signals 426 may travel from peripheral nerves of the patient, through the first spinal nerve 430, to the dorsal horn 422 of the spinal cord 120. Additionally, or alternatively, one or more signals 428 may travel from the ventral horn 424 of the spinal cord 120 to peripheral nerves of the patient 105.

An epidural space 460 may exist between a vertebral bone of the spinal column 400 and the spinal cord 120. Epidural space 460 may include fat without including nerve tissue and bone. Epidural space 460 may extend along at least a portion of spinal cord 120. Consequently, it may be beneficial to implant one or more leads within the epidural space so that the one or more leads can deliver electrical stimulation to the spinal cord 120 and other nerve tissue proximate to the spinal cord 120 (e.g., dorsal root 432). The epidural space 460 may include a dorsal space and a ventral space. The dorsal space may represent a portion of the epidural space that is on a dorsal side of the spinal cord 120. The ventral space may represent a portion of the epidural space that is on a ventral side of the spinal cord 120.

A first lead position 482 and a second lead position 484 are illustrated in FIG. 4, but the techniques described herein are not meant to be limited to leads being placed in the first lead position 482 and the second lead position 484. Leads may be placed in other positions not illustrated in FIG. 4. The dorsal space of the epidural space 460 may include a central region 462, a first lateral region 464, and a second lateral region 466. As seen in FIG. 4, the first lead position 482 is within the first lateral region 464, and the second lead position 484 is within the central region 462. It may be beneficial for a clinician to place a lead in the first lead position 482 within the first lateral region 464 during implant, so that IMD 200 may detect one or more bioelectric signals corresponding to dorsal root 432. For example, the one or more signals 426 travelling from peripheral nerves to the dorsal horn 422 may include one or more pain signals. A lead in the first lead position 482 may be configured to sense bioelectric signals corresponding to the pain signals travelling through dorsal root 432 more effectively than a lead in the second lead position 484 can sense bioelectric signals corresponding to the pain signals travelling through dorsal root 432, because the first lead position 482 is closer to dorsal root 432 than the second lead position 484. Moreover, the first lateral region 464 of the epidural space 460 is narrower than the central region 462, so a smaller amount of fat separates electrodes of a lead in the first lead position 482 from nerve tissue than separates electrodes of a lead in the second lead position 484 from nerve tissue. Consequently, bioelectric signals sensed via a lead in the first lead position 482 may include a smaller amount of noise as compared with bioelectric signals sensed via a lead in the first lead position 482.

In some examples, a clinician may place a lead (e.g., lead 130A of FIG. 1) in the first lead position 482 during an implant of leads 130. IMD 110 may sense, via electrodes of lead 130A, one or more bioelectric signals corresponding to the one or more signals 426 travelling through dorsal root 432 to dorsal horn 422. For example, IMD 110 may detect one or more LFPs and/or one or more ECAPs corresponding to the signals 426. In some examples, the one or more bioelectric signals may include a set of bioelectric signal components, wherein each bioelectric signal component of the set of bioelectric signal components corresponds to a respective electrode of one or more electrodes on lead 130A. For example, lead 130A may include one or more electrodes placed along the length of the lead. Since FIG. 4 is a cross-section of the epidural space, lead 130A may extend through the first lead position 482 out of the plane of FIG. 4 and into the plane of FIG. 4. Some of the electrodes may be displaced above the plane of FIG. 4 and some of the electrodes may be displaced below the plane of FIG. 4. In some examples, an electrode may be located on the plane of FIG. 4. In any case, IMD 110 may be configured to determine, based on the bioelectric signal corresponding to each electrode of the one or more electrodes, a proximity of each electrode of the one or more electrodes to the dorsal root 432.

In some examples, a clinician may place a lead (e.g., lead 130B of FIG. 1) in the second lead position 484 during an implant of leads 130. When the one or more bioelectric signals sensed via lead 130A include ECAPs, the ECAPs may, in some cases, be elicited by lead 130B located in the second lead position 484. For example, IMD 110 may deliver a sequence of stimulation pulses via lead 130B, eliciting one or more ECAPs that travel from the dorsal horn 422 through the dorsal root 432. IMD 110 may sense the one or more ECAPs via the lead 130A located in the first lead position 482, and determine the proximity of each electrode of the one or more electrodes on the lead 130A to the dorsal root 432. Additionally, or alternatively, when the one or more bioelectric signals sensed via lead 130A include ECAPs, the ECAPs may, in some cases, be elicited by lead 130A located in the first lead position 482. That is, Lead 130A may both deliver electrical stimulation pulses that elicit ECAPs and sense the ECAPs.

In some examples, a clinician may move a lead along the epidural space 460 while keeping the lead in the first lead position 482. That is, the lead may remain in the same position in the epidural space 460 relative to the cross section illustrated in FIG. 4, but the lead may move upwards or downwards relative to the plane of the cross section. As a result, electrodes on the lead may move upwards or downwards relative to the plane of the cross section. IMD 110 may output information indicative of the proximity of the electrodes to dorsal root 432 in real time, so that the clinician can place the electrodes proximate to the dorsal root 432 based on the information output by IMD 110. In addition, or alternatively, these techniques may be used to determine the location of already implanted electrodes. Therefore, a clinician or system may determine, based on the sensed bioelectric signals, which electrodes to use for subsequent stimulation and/or sensing of signals from the patient.

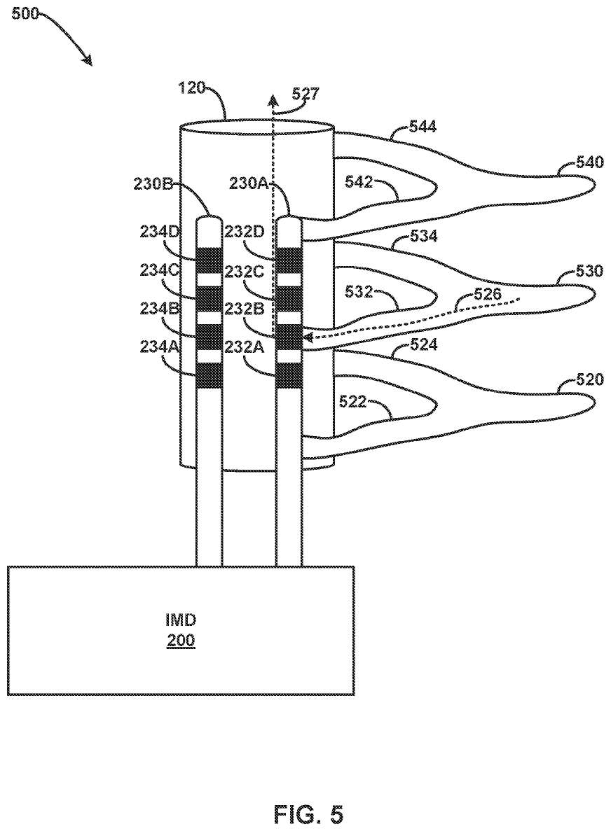

FIG. 5 is a conceptual diagram illustrating a perspective view of an example spinal column 500, in accordance with one or more techniques of this disclosure. Spinal column 500 includes spinal cord 120. Spinal column 500 may also include vertebra bones (not illustrated in FIG. 5). Spinal cord 120 may include one or more nerves that carry signals to and from a brain of patient 105. As seen in FIG. 5, spinal nerve 520, spinal nerve 530, and spinal nerve 540 may be connected to spinal cord 120. For example, spinal nerve 520 may include a dorsal root 522 and a ventral root 524 connected to spinal cord 120, spinal nerve 530 may include a dorsal root 532 and a ventral root 534 connected to spinal cord 120, and spinal nerve 540 may include a dorsal root 532 and a ventral root 534 connected to spinal cord 120. Spinal nerve 530 may be an example of spinal nerve 430 of FIG. 4. Dorsal root 532 may be an example of dorsal root 432 of FIG. 4. Ventral root 534 may be an example of ventral root 434 of FIG. 4.

Lead 230A and lead 230B may be implanted in an epidural space of the patient 105. In some examples, lead 230A may be implanted proximate to a location where dorsal root 532 connects with spinal cord 120. Lead 230A includes electrodes 232A-232D spaced along a body of lead 230A. Consequently, each electrode of electrodes 232A-232D is located in a different position relative to dorsal root 532. As seen in FIG. 5, electrode 232B is closest to dorsal root 532 and electrode 232D is farthest away from dorsal root 532. IMD 200 may receive one or more bioelectric signals (e.g., LFPs and/or ECAPs). Additionally, IMD 200 may determine, based on the one or more bioelectric signals, a proximity of each electrode of electrodes 232A-232D to dorsal root 532. In some examples, IMD 200 may sense the one or more bioelectric signals based on signals 526 travelling through dorsal root 532. For example, signals 536 may cause IMD 200 to sense one or more LFPs. Additionally, or alternatively, one or both of electrodes 232A and 232B may deliver a sequence of stimulation pulses eliciting one or more ECAPs, where the one or more ECAPs indicate characteristics of the signals 526 travelling through dorsal root 532. IMD 200 may sense the one or more ECAPs via lead 230A. In some examples, spinal cord 120 may receive the signals 526 and transmit signals 527 to the brain. When signals 526 include pain signals, signals 527 may carry the pain signals to the brain and cause the patient to experience pain.

In some examples, IMD 200 may output, in real time or near-real time, information indicative one the proximity of each electrode of electrodes 232A-232D to the dorsal root 532 for display by a user interface. A clinician may adjust a position of lead 230A during implant based on the information indicating the proximity of each electrode of electrodes 232A-232D. For example, a clinician may adjust the position of lead 230A downwards along a longitudinal axis of spinal cord 120 so that electrodes 232A is closest to dorsal root 532.

FIG. 6 is a flow diagram illustrating an example operation for determining a position of one or more electrodes based on sensed bioelectric signals, in accordance with one or more techniques of this disclosure. For convenience, FIG. 6 is described with respect to IMD 200 and leads 230 of FIG. 2. However, the techniques of FIG. 6 may be performed by different components of IMD 200 or by additional or alternative medical devices.

IMD 200 may detect one or more bioelectric signals of patient 105 (602). In some examples, IMD 200 may detect the one or more bioelectric signals via a lead that is implanted in a lateral region of the epidural space. The one or more bioelectric signals may include one or both of ECAPs and LFPs. IMD 200 may identify a set of bioelectric signal components in the one or more bioelectric signals (604). In some examples, each bioelectric signal component of the set of bioelectric signal components may correspond to a respective electrode of a set of electrodes located on the lead that is implanted in the lateral region of the epidural space. IMD 200 may determine, based on the set of bioelectric signal components, a proximity of each electrode of the set of electrodes on the lead implanted in the epidural space to a dorsal root of the patient 105 (606). In some examples, the dorsal root may carry pain signals to the spinal cord of the patient 105. IMD 200 may output information indicative of the proximity of each electrode of the set of electrodes to the dorsal root (608).

The following clauses are example systems, devices, and methods described herein.

Clause 1: A system including sensing circuitry configured to detect, via a lead, a bioelectric signal of the patient, wherein the lead comprises one or more electrodes, and wherein the lead is configured to be located in an epidural space of a patient and displaced laterally from a dorsal horn of the patient; and processing circuitry configured to: determine, based on the bioelectric signal, a proximity of each electrode of the one or more electrodes to a dorsal root of the patient; and output information indicative of the proximity of each electrode of the one or more electrodes to the dorsal root.

Clause 2: The system of clause 1, wherein the bioelectric signal is indicative of one or more characteristics of a pain signal carried from the dorsal root to the dorsal horn of the patient, and wherein to determine the proximity of each electrode of the one or more electrodes to the dorsal root, the processing circuitry is configured to determine the proximity of each electrode of the one or more electrodes to the dorsal root carrying the pain signal.

Clause 3: The system of any of clauses 1-2, wherein the bioelectric signal comprises a local field potential (LFP).

Clause 4: The system of any of clauses 1-3, wherein the bioelectric signal comprises one or more evoked compound action potentials (ECAPs), and wherein the system further comprises stimulation generation circuitry configured to deliver electrical stimulation to the patient, wherein the electrical stimulation comprises a plurality of pulses, and wherein one or more pulses of the plurality of pulses is configured to elicit a respective ECAP of the one or more ECAPs.

Clause 5: The system of clause 4, wherein the lead is a first lead, wherein the one or more electrodes are a first one or more electrodes, wherein the sensing circuitry is configured to detect the bioelectric signal via the first one or more electrodes, and wherein the stimulation generation circuitry is configured to deliver the electrical stimulation to the patient via a second one or more electrodes of a second lead comprising the second one or more electrodes, wherein the second lead is configured to be located in the epidural space of the patient proximate to the dorsal horn of the patient.

Clause 6: The system of any of clauses 4-5, wherein the sensing circuitry is configured to detect the bioelectric signal via the one or more electrodes, and wherein the stimulation generation circuitry is configured to deliver the electrical stimulation to the patient via the one or more electrodes.

Clause 7: The system of any of clauses 1-6, wherein to detect the bioelectric signal of the patient, the sensing circuitry is configured to: detect a set of bioelectric signal components, wherein each bioelectric signal component of the set of bioelectric signal components corresponds to a respective electrode of the one or more electrodes, and determine the proximity of each electrode of the one or more electrodes to the dorsal root by at least: identifying one or more characteristics of each bioelectric signal component of the set of bioelectric signal components; and determining, based on the one or more characteristics of each bioelectric signal component of the set of bioelectric signal components, the proximity of each electrode of the one or more electrodes to the dorsal root.

Clause 8: The system of any of clauses 1-7, wherein the bioelectric signal is a first bioelectric signal, and wherein the sensing circuitry is configured to: detect the first bioelectric signal when the lead is placed in a first location of the epidural space; and detect a second bioelectric signal of the patient when the lead is placed in a second location of the epidural space, the second location being displaced from the first location along a longitudinal axis of the epidural space, and wherein the processing circuitry is further configured to: determine, based on the second bioelectric signal, a proximity of each electrode of the one or more electrodes to the dorsal root of the patient when the lead is placed in the second location; and output information indicative of the proximity of each electrode of the one or more electrodes to the dorsal root when the lead is placed in the second location.

Clause 9: The system of any of clauses 1-8, wherein the processing circuitry is further configured to: determine, based on the bioelectric signal prior to delivering electrical stimulation, a first activity level corresponding to the dorsal root of the patient; receive information indicative of a first pain level of the patient prior to delivering electrical stimulation; control the medical device to deliver electrical stimulation to the patient via the one or more electrodes; determine, based on the bioelectric signal after delivering electrical stimulation, a second activity level corresponding to the dorsal root of the patient; receive information indicative of a second pain level of the patient after delivering electrical stimulation; and determine, based on the first activity level the second activity level, the first pain level, and the second pain level, a third activity level, wherein the third activity level represents a threshold where the patient begins to experience uncomfortable pain.

Clause 10: The system of any of clauses 1-9, wherein the system comprises an implantable medical device that comprises the processing circuitry and the sensing circuitry.

Clause 11: A method comprising: detecting, by sensing circuitry via a lead, a bioelectric signal of the patient, wherein the lead comprises one or more electrodes, and wherein the lead is configured to be located in an epidural space of a patient and displaced laterally from a dorsal horn of the patient; determining, by processing circuitry based on the bioelectric signal, a proximity of each electrode of the one or more electrodes to a dorsal root of the patient; and outputting, by the processing circuitry, information indicative of the proximity of each electrode of the one or more electrodes to the dorsal root.

Clause 12: The method of clause 11, wherein the bioelectric signal is indicative of one or more characteristics of a pain signal carried from the dorsal root to the dorsal horn of the patient, and wherein determining the proximity of each electrode of the one or more electrodes to the dorsal root comprises determining, by the processing circuitry, the proximity of each electrode of the one or more electrodes to the dorsal root carrying the pain signal.

Clause 13: The method of any of clauses 11-12, wherein the bioelectric signal comprises a local field potential (LFP).

Clause 14: The method of any of clauses 11-13, wherein the bioelectric signal comprises one or more evoked compound action potentials (ECAPs), and wherein the method further comprises delivering, by stimulation generation circuitry, electrical stimulation to the patient, wherein the electrical stimulation comprises a plurality of pulses, and wherein one or more pulses of the plurality of pulses is configured to elicit a respective ECAP of the one or more ECAPS.

Clause 15: The method of clause 14, wherein the lead is a first lead, wherein the one or more electrodes are a first one or more electrodes, and wherein the method further comprises: detecting, by the sensing circuitry, the bioelectric signal via the first one or more electrodes; and delivering, by the stimulation generation circuitry, the electrical stimulation to the patient via a second one or more electrodes of a second lead comprising the second one or more electrodes, wherein the second lead is configured to be located in the epidural space of the patient proximate to the dorsal horn of the patient.

Clause 16: The method of any of clauses 14-15, further comprising: detecting, by the sensing circuitry, the bioelectric signal via the one or more electrodes; and delivering, by the stimulation generation circuitry, the electrical stimulation to the patient via the one or more electrodes.

Clause 17: The method of any of clauses 11-16, wherein detecting the bioelectric signal of the patient comprises: detecting, by the sensing circuitry, a set of bioelectric signal components, wherein each bioelectric signal component of the set of bioelectric signal components corresponds to a respective electrode of the one or more electrodes; and determining the proximity of each electrode of the one or more electrodes to the dorsal root by at least: identifying one or more characteristics of each bioelectric signal component of the set of bioelectric signal components; and determining, based on the one or more characteristics of each bioelectric signal component of the set of bioelectric signal components, the proximity of each electrode of the one or more electrodes to the dorsal root.

Clause 18: The method of any of clauses 11-17, wherein the bioelectric signal is a first bioelectric signal, and wherein the method further comprises: detecting, by the sensing circuitry, the first bioelectric signal when the lead is placed in a first location of the epidural space; and detecting, by the sensing circuitry, a second bioelectric signal of the patient when the lead is placed in a second location of the epidural space, the second location being displaced from the first location along a longitudinal axis of the epidural space, and wherein the method further comprises: determining, by the processing circuitry based on the second bioelectric signal, a proximity of each electrode of the one or more electrodes to the dorsal root of the patient when the lead is placed in the second location; and outputting, by the processing circuitry, information indicative of the proximity of each electrode of the one or more electrodes to the dorsal root when the lead is placed in the second location.

Clause 19: The method of any of clauses 11-18, further comprising: determining, by the processing circuitry based on the bioelectric signal prior to delivering electrical stimulation, a first activity level corresponding to the dorsal root of the patient; receiving, by the processing circuitry, information indicative of a first pain level of the patient prior to delivering electrical stimulation; controlling, by the processing circuitry, the medical device to deliver electrical stimulation to the patient via the one or more electrodes; determining, by the processing circuitry based on the bioelectric signal after delivering electrical stimulation, a second activity level corresponding to the dorsal root of the patient; receiving, by the processing circuitry, information indicative of a second pain level of the patient after delivering electrical stimulation; and determining, by the processing circuitry based on the first activity level the second activity level, the first pain level, and the second pain level, a third activity level, wherein the third activity level represents a threshold where the patient begins to experience uncomfortable pain.

Clause 20: A computer-readable storage medium comprising instructions that, when executed, cause one or more processors to: detect, via a lead, a bioelectric signal of the patient, wherein the lead comprises one or more electrodes, and wherein the lead is configured to be located in an epidural space of a patient and displaced laterally from a dorsal horn of the patient; determine, based on the bioelectric signal, a proximity of each electrode of the one or more electrodes to a dorsal root of the patient; and output information indicative of the proximity of each electrode of the one or more electrodes to the dorsal root.

The techniques described in this disclosure may be implemented, at least in part, in hardware, software, firmware or any combination thereof. For example, various aspects of the described techniques may be implemented within one or more processors or processing circuitry, including one or more microprocessors, DSPs, ASICs, FPGAs, or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, circuits or components may be implemented together or separately as discrete but interoperable logic devices. Depiction of different features as circuits or units is intended to highlight different functional aspects and does not necessarily imply that such circuits or units must be realized by separate hardware or software components. Rather, functionality associated with one or more circuits or units may be performed by separate hardware or software components or integrated within common or separate hardware or software components.

The techniques described in this disclosure may also be embodied or encoded in a computer-readable medium, such as a computer-readable storage medium, containing instructions that may be described as non-transitory media. Instructions embedded or encoded in a computer-readable storage medium may cause a programmable processor, or other processor, to perform the method, e.g., when the instructions are executed. Computer readable storage media may include RAM, ROM, PROM, EPROM, EEPROM, flash memory, a hard disk, a CD-ROM, a floppy disk, a cassette, magnetic media, optical media, or other computer readable media.

Various examples have been described. These and other examples are within the scope of the following claims.

1. A system comprising:
sensing circuitry configured to detect, via a lead, a bioelectric signal of the patient, wherein the lead comprises one or more electrodes, and wherein the lead is configured to be located in an epidural space of a patient and displaced laterally from a dorsal horn of the patient; and
processing circuitry configured to:
determine, based on the bioelectric signal, a proximity of each electrode of the one or more electrodes to a dorsal root of the patient; and
output information indicative of the proximity of each electrode of the one or more electrodes to the dorsal root.

2. The system of claim 1, wherein the bioelectric signal is indicative of one or more characteristics of a pain signal carried from the dorsal root to the dorsal horn of the patient, and wherein to determine the proximity of each electrode of the one or more electrodes to the dorsal root, the processing circuitry is configured to determine the proximity of each electrode of the one or more electrodes to the dorsal root carrying the pain signal.

3. The system of claim 1, wherein the bioelectric signal comprises a local field potential (LFP).

4. The system of claim 1, wherein the bioelectric signal comprises one or more evoked compound action potentials (ECAPs), and wherein the system further comprises stimulation generation circuitry configured to deliver electrical stimulation to the patient, wherein the electrical stimulation comprises a plurality of pulses, and wherein one or more pulses of the plurality of pulses is configured to elicit a respective ECAP of the one or more ECAPs.

5. The system of claim 4, wherein the lead is a first lead, wherein the one or more electrodes are a first one or more electrodes, wherein the sensing circuitry is configured to detect the bioelectric signal via the first one or more electrodes, and wherein the stimulation generation circuitry is configured to deliver the electrical stimulation to the patient via a second one or more electrodes of a second lead comprising the second one or more electrodes, wherein the second lead is configured to be located in the epidural space of the patient proximate to the dorsal horn of the patient.

6. The system of claim 1, wherein the sensing circuitry is configured to detect the bioelectric signal via the one or more electrodes, and wherein the stimulation generation circuitry is configured to deliver the electrical stimulation to the patient via the one or more electrodes.

7. The system of claim 1, wherein to detect the bioelectric signal of the patient, the sensing circuitry is configured to:
detect a set of bioelectric signal components, wherein each bioelectric signal component of the set of bioelectric signal components corresponds to a respective electrode of the one or more electrodes, and
determine the proximity of each electrode of the one or more electrodes to the dorsal root by at least:
identifying one or more characteristics of each bioelectric signal component of the set of bioelectric signal components; and
determining, based on the one or more characteristics of each bioelectric signal component of the set of bioelectric signal components, the proximity of each electrode of the one or more electrodes to the dorsal root.

8. The system of claim 1, wherein the bioelectric signal is a first bioelectric signal, and wherein the sensing circuitry is configured to:
detect the first bioelectric signal when the lead is placed in a first location of the epidural space; and
detect a second bioelectric signal of the patient when the lead is placed in a second location of the epidural space, the second location being displaced from the first location along a longitudinal axis of the epidural space, and
wherein the processing circuitry is further configured to:
determine, based on the second bioelectric signal, a proximity of each electrode of the one or more electrodes to the dorsal root of the patient when the lead is placed in the second location; and
output information indicative of the proximity of each electrode of the one or more electrodes to the dorsal root when the lead is placed in the second location.

9. The system of claim 1, wherein the processing circuitry is further configured to:
determine, based on the bioelectric signal prior to delivering electrical stimulation, a first activity level corresponding to the dorsal root of the patient;
receive information indicative of a first pain level of the patient prior to delivering electrical stimulation;
control the medical device to deliver electrical stimulation to the patient via the one or more electrodes;
determine, based on the bioelectric signal after delivering electrical stimulation, a second activity level corresponding to the dorsal root of the patient;
receive information indicative of a second pain level of the patient after delivering electrical stimulation; and
determine, based on the first activity level the second activity level, the first pain level, and the second pain level, a third activity level, wherein the third activity level represents a threshold where the patient begins to experience uncomfortable pain.

10. The system of claim 1, wherein the system comprises an implantable medical device that comprises the processing circuitry and the sensing circuitry.

11. A method comprising:
detecting, by sensing circuitry via a lead, a bioelectric signal of the patient, wherein the lead comprises one or more electrodes, and wherein the lead is configured to be located in an epidural space of a patient and displaced laterally from a dorsal horn of the patient;
determining, by processing circuitry based on the bioelectric signal, a proximity of each electrode of the one or more electrodes to a dorsal root of the patient; and
outputting, by the processing circuitry, information indicative of the proximity of each electrode of the one or more electrodes to the dorsal root.

12. The method of claim 11, wherein the bioelectric signal is indicative of one or more characteristics of a pain signal carried from the dorsal root to the dorsal horn of the patient, and wherein determining the proximity of each electrode of the one or more electrodes to the dorsal root comprises determining, by the processing circuitry, the proximity of each electrode of the one or more electrodes to the dorsal root carrying the pain signal.

13. The method of claim 1, wherein the bioelectric signal comprises a local field potential (LFP).

14. The method of claim 1, wherein the bioelectric signal comprises one or more evoked compound action potentials (ECAPs), and wherein the method further comprises delivering, by stimulation generation circuitry, electrical stimulation to the patient, wherein the electrical stimulation comprises a plurality of pulses, and wherein one or more pulses of the plurality of pulses is configured to elicit a respective ECAP of the one or more ECAPs.

15. The method of claim 14, wherein the lead is a first lead, wherein the one or more electrodes are a first one or more electrodes, and wherein the method further comprises:
detecting, by the sensing circuitry, the bioelectric signal via the first one or more electrodes; and
delivering, by the stimulation generation circuitry, the electrical stimulation to the patient via a second one or more electrodes of a second lead comprising the second one or more electrodes, wherein the second lead is configured to be located in the epidural space of the patient proximate to the dorsal horn of the patient.

16. The method of claim 1, further comprising:
detecting, by the sensing circuitry, the bioelectric signal via the one or more electrodes; and
delivering, by the stimulation generation circuitry, the electrical stimulation to the patient via the one or more electrodes.

17. The method of claim 1, wherein detecting the bioelectric signal of the patient comprises:
detecting, by the sensing circuitry, a set of bioelectric signal components, wherein each bioelectric signal component of the set of bioelectric signal components corresponds to a respective electrode of the one or more electrodes; and
determining the proximity of each electrode of the one or more electrodes to the dorsal root by at least:
identifying one or more characteristics of each bioelectric signal component of the set of bioelectric signal components; and
determining, based on the one or more characteristics of each bioelectric signal component of the set of bioelectric signal components, the proximity of each electrode of the one or more electrodes to the dorsal root.

18. The method of claim 1, wherein the bioelectric signal is a first bioelectric signal, and wherein the method further comprises:
detecting, by the sensing circuitry, the first bioelectric signal when the lead is placed in a first location of the epidural space; and
detecting, by the sensing circuitry, a second bioelectric signal of the patient when the lead is placed in a second location of the epidural space, the second location being displaced from the first location along a longitudinal axis of the epidural space, and
wherein the method further comprises:
determining, by the processing circuitry based on the second bioelectric signal, a proximity of each electrode of the one or more electrodes to the dorsal root of the patient when the lead is placed in the second location; and
outputting, by the processing circuitry, information indicative of the proximity of each electrode of the one or more electrodes to the dorsal root when the lead is placed in the second location.

19. The method of claim 1, further comprising:
determining, by the processing circuitry based on the bioelectric signal prior to delivering electrical stimulation, a first activity level corresponding to the dorsal root of the patient;
receiving, by the processing circuitry, information indicative of a first pain level of the patient prior to delivering electrical stimulation;
controlling, by the processing circuitry, the medical device to deliver electrical stimulation to the patient via the one or more electrodes;
determining, by the processing circuitry based on the bioelectric signal after delivering electrical stimulation, a second activity level corresponding to the dorsal root of the patient;
receiving, by the processing circuitry, information indicative of a second pain level of the patient after delivering electrical stimulation; and
determining, by the processing circuitry based on the first activity level the second activity level, the first pain level, and the second pain level, a third activity level, wherein the third activity level represents a threshold where the patient begins to experience uncomfortable pain.

20. A computer-readable storage medium comprising instructions that, when executed, cause
one or more processors to:
detect, via a lead, a bioelectric signal of the patient, wherein the lead comprises one or more electrodes, and wherein the lead is configured to be located in an epidural space of a patient and displaced laterally from a dorsal horn of the patient;
determine, based on the bioelectric signal, a proximity of each electrode of the one or more electrodes to a dorsal root of the patient; and
output information indicative of the proximity of each electrode of the one or more electrodes to the dorsal root.
